(19) United States
(12) Patent Application Publication
(10) Pub. No.: US 2017/0251992 A1
(43) Pub. Date: Sep. 7, 2017

(54) X-RAY APPARATUS AND METHOD OF SCANNING THE SAME

(71) Applicant: Samsung Electronics Co., Ltd, Gyeonggi-do (KR)

(72) Inventors: Woo-young Jang, Gyeonggi-do (KR); Hyeon-min Lee, Gyeonggi-do (KR); Ho-seong Kwak, Seoul (KR); Jae-guyn Lim, (US); Woo-sup Han, Gyeonggi-do (KR)

(73) Assignee: Samsung Elecatronics Co., Ltd., Gyeonggi-do (KR)

(21) Appl. No.: 15/511,227

(22) PCT Filed: Jun. 24, 2015
(86) PCT No.: PCT/KR2015/006396

§ 371 Date: Mar. 14, 2017

(30) Foreign Application Priority Data
Sep. 18, 2014 (KR) 10-2014-0124630

(51) Int. Cl. A61B 6/00; A61B 6/12; A61B 6/04

(52) U.S. Cl. CPC A61B 6/541; A61B 6/0407; A61B 6/0457; A61B 6/12; A61B 6/4441; A61B 6/469; A61B 6/487; A61B 6/4476

(57) ABSTRACT

An X-ray apparatus includes a C-arm for adjusting a position of an X-ray source; a table on which an object is positioned; a data obtaining unit for obtaining position information of a target in the object; and a control unit for moving at least one of the C-arm and the table to allow tracking of the target based on the position information when capturing an X-ray image.

### TECHNICAL FIELD

One or more exemplary embodiments relate to an X-ray apparatus and a method of scanning the X-ray apparatus, and more particularly, to an X-ray apparatus that moves at least one of a C-arm and a table in order to allow tracking of a target when capturing an X-ray image.

### BACKGROUND ART

An X-ray apparatus is a medical imaging apparatus that acquires images of internal structures of the human body by transmitting an X-ray through the human body. The X-ray apparatus may acquire medical images of an object more simply within a shorter time than other medical imaging apparatuses including a magnetic resonance imaging (MRI) apparatus and a computed tomography (CT) apparatus. Therefore, the X-ray apparatus is widely used in general chest scanning, abdomen scanning, skeleton scanning, nasal sinuses scanning, neck soft tissue scanning, and breast scanning.

Fluoroscopy refers to an image processing technique of acquiring an X-ray video by scanning an object in real time. A user may use fluoroscopy in order to monitor X-ray angiography, surgical treatment or the like.

X-ray scanning including fluoroscopy image scanning uses radiation, and thus, a user has to adjust a dose of radiation that an object is exposed to. In particular, fluoroscopy requires X-ray scanning for a relatively long period of time, and thus various techniques to minimize the dose of radiation are being developed. For example, one technique involves obtaining a plurality of low-quality frames by X-ray scanning using a low dose of radiation and combining the low-quality frames to restore image quality. In addition, there is a dynamic region of interest (ROI) technique capable of minimizing the dose of radiation by radiating an X-ray only to regions around a target while tracking the target.

However, the related art merely involves easily informing a user about a position of a target in an X-ray image. Thus, if the target goes beyond a fluoroscopy image, there is an inconvenience in that a user himself/herself needs to adjust a position of the C-arm or table. Accordingly, there is a problem in that a surgery time increases, and thus a dose of radiation radiated onto an object unnecessarily increases.

Thus, in regard to X-ray scanning or fluoroscopy image scanning, an X-ray apparatus is required that is capable of minimizing an amount of radiation that an object is exposed to and more efficiently adjusting a region to which the X-ray is radiated in the object according to a user's intention so that the user may further concentrate on surgery.

### DISCLOSURE OF INVENTION

### Solution to Problem

According to one or more exemplary embodiments, an X-ray apparatus includes a C-arm for adjusting a position of an X-ray source; a table on which an object is positioned; a data obtaining unit for obtaining position information of a target in the object; and a control unit for moving at least one of the C-arm and the table to allow tracking of the target based on the position information when capturing an X-ray image.

### Advantageous Effects of Invention

One or more exemplary embodiments include an X-ray apparatus capable of minimizing a dose of radiation that an object is exposed to by moving at least one of a C-arm and a table based on a position of a target.

In more detail, at least one of the C-arm and the table may be automatically moved based on position information of the target such that an X-ray may be exactly radiated onto the target that is moving or is not moving. One or more exemplary embodiments include an X-ray apparatus capable of minimizing a test time or a surgery time and minimizing a dose of radiation radiated onto the target and a method of X-ray scanning.

Additional aspects will be set forth in part in the description which follows and, in part, will be apparent from the description, or may be learned by practice of the presented exemplary embodiments.

### BRIEF DESCRIPTION OF DRAWINGS

These and/or other aspects will become apparent and more readily appreciated from the following description of the exemplary embodiments, taken in conjunction with the accompanying drawings, in which:

FIG. 1 is a block diagram of an X-ray apparatus according to an exemplary embodiment;

FIGS. 2A through 2C are diagrams of C-arm X-ray apparatuses to which exemplary embodiments are applicable;

FIGS. 3A through 3D are diagrams for explaining operations of C-arms of an X-ray apparatus according to an exemplary embodiment;

FIGS. 4A through 4C are diagrams for explaining operations of tables of an X-ray apparatus according to an exemplary embodiment;

FIG. 5 is a block diagram of an X-ray apparatus according to an exemplary embodiment;

FIG. 6 is a block diagram of an X-ray apparatus according to another exemplary embodiment;

FIGS. 7A through 7C are diagrams for explaining operations of an X-ray apparatus according to an exemplary embodiment;

FIGS. 8A through 8D are diagrams for explaining operations of an X-ray apparatus according to another exemplary embodiment;

FIGS. 9A through 9E are diagrams for explaining operations of an X-ray apparatus according to another exemplary embodiment;

FIGS. 10A through 10E are diagrams for explaining operations of an X-ray apparatus according to another exemplary embodiment;

FIGS. 11A through 11E are diagrams for explaining operations of an X-ray apparatus according to another exemplary embodiment;

FIGS. 12A through 12E are diagrams for explaining operations of an X-ray apparatus according to another exemplary embodiment;

FIGS. 13A through 13E are diagrams for explaining operations of an X-ray apparatus according to another exemplary embodiment;

FIG. 14 is a diagram for explaining an operation of an X-ray apparatus according to an exemplary embodiment;

FIG. 15 is a diagram for explaining an operation of an X-ray apparatus according to another exemplary embodiment;

FIGS. 16A and 16B are diagrams for explaining operations of an X-ray apparatus according to an exemplary embodiment;

FIGS. 17A and 17B are diagrams for explaining operations of an X-ray apparatus according to another exemplary embodiment;

FIG. 18 is a diagram for explaining operations of X-ray apparatuses according to an exemplary embodiment;

FIG. 19 is a diagram for explaining operations of X-ray apparatuses according to another exemplary embodiment;

FIG. 20 is a diagram for explaining operations of X-ray apparatuses according to another exemplary embodiment;

FIG. 21 is a diagram for explaining operations of X-ray apparatuses according to another exemplary embodiment;

FIG. 22 is a diagram for explaining operations of X-ray apparatuses according to another exemplary embodiment;

FIG. 23 is a diagram for explaining operations of X-ray apparatuses according to another exemplary embodiment;

FIG. 24 is a diagram for explaining operations of X-ray apparatuses according to another exemplary embodiment; and

FIG. 25 is a flowchart of an X-ray scanning method according to an exemplary embodiment.

### BEST MODE FOR CARRYING OUT THE INVENTION

According to one or more exemplary embodiments, an X-ray apparatus includes a C-arm for adjusting a position of an X-ray source; a table on which an object is positioned; a data obtaining unit for obtaining position information of a target in the object; and a control unit for moving at least one of the C-arm and the table to allow tracking of the target based on the position information when capturing an X-ray image.

The control unit may move at least one of the C-arm and the table to allow the target to be positioned within a predetermined distance from the center of the X-ray image based on the position information.

The control unit may move at least one of the C-arm and the table to allow the target to be positioned in the center of the X-ray image based on the position information.

The control unit may move at least one of the C-arm and the table to allow tracking of at least one of the target and a region of interest (ROI) based on the position information when capturing the X-ray image.

The control unit may allow the target to be positioned within a predetermined distance from the center of the X-ray image based on the position information, recognize a boundary between the object and the target, and move at least one of the C-arm and the table to track an ROI based on the recognized boundary.

The C-arm may adjust a position of the X-ray source through at least one of a longitudinal motion, a lateral motion, a tilting motion, a rotational motion, and a spherical motion.

The table may adjust a position of the object through at least one of a longitudinal motion, a lateral motion, a tilting motion, a rotational motion, and a spherical motion.

The X-ray apparatus may further include: a user interface unit for receiving a first input selecting at least one of the C-arm and the table to be moved and setting an order of movements of the selected at least one of the C-arm and table, wherein the control unit moves at least one of the C-arm and the table to allow tracking of the target based on the first input and the position information when capturing the X-ray image.

The C-arm may adjust a position of the X-ray source through at least one of a longitudinal motion, a lateral motion, a tilting motion, a rotational motion, and a spherical motion, wherein the table adjusts a position of the object through at least one of a longitudinal motion, a lateral motion, a tilting motion, and a rotational motion. The X-ray apparatus may further include: a user interface unit for selecting at least one of the motions of the C-arm and the motions of the table to be controlled and receiving a second input setting a control order of the selected motions, wherein the control unit moves at least one of the C-arm and the table to allow tracking of the target based on the second input and the position information when capturing the X-ray image.

The X-ray apparatus may further include: a user interface unit for receiving a third input for stopping a movement of at least one of the C-arm and the table, wherein the control unit stops the movement of at least one of the C-arm and the table based on the third input.

The X-ray source may emit an X-ray to the object. The X-ray apparatus may further include: a detection unit for detecting the X-ray penetrating the object, wherein the C-arm connects the X-ray source and the detection unit and adjusts positions of the X-ray source and the detection unit.

The target may be a tip of a catheter.

The X-ray image may be a fluoroscopy image.

The data obtaining unit may obtain the position information of the target based on electrode signals detected from a plurality of electrodes attached to the object.

The control unit may set first coordinates indicating a position of the target on a first coordinate system regarding the object based on the position information, transform the first coordinates into second coordinates on a second coordinate system regarding the X-ray image, and move at least one of the C-arm and the table to track the target based on the second coordinates.

The control unit may set the first coordinate system as a 3-dimensional (3D) rectangular coordinate system based on electrode signals detected from a plurality of electrodes attached to positions corresponding to three axes that are perpendicular to each other, sets the second coordinate system as a 2D rectangular coordinate system that is a plane perpendicular to an irradiation direction of an X-ray, and set a point on the plane closest to the first coordinates as the second coordinates.

The control unit may set the first coordinate system as a 3D rectangular coordinate system based on electrode signals detected from a plurality of electrodes attached to positions corresponding to three axes that are perpendicular to each other, and set the second coordinate system as a 3D rectangular coordinate system including an axis in the same direction as an irradiation direction of an X-ray.

The control unit may set first coordinates indicating a position of the target on a first coordinate system regarding the object and transforms the first coordinates into second coordinates on a second coordinate system regarding the X-ray image based on an angle of the C-arm and the position information.

The data obtaining unit may include a plurality of electrocardiogram (ECG) measurement electrodes attached to the object, wherein the electrode signals are ECG signals.

The data obtaining unit may measure impedance of the object included in an ROI based on the electrode signals and obtain the position information based on the impedance of the object.

The data obtaining unit may measure impedance of the object included in an ROI based on the electrode signals, generate an impedance map of the object based on the impedance of the object, and obtain the position information based on the impedance map of the object.

The data obtaining unit may obtain position information of the target in the object by image tracking the target appearing in the X-ray image.

According to one or more exemplary embodiments, an X-ray apparatus includes a C-arm for adjusting a position of an X-ray source; a data obtaining unit for obtaining position information of a target in an object; and a control unit for moving the C-arm to allow tracking of the target based on the position information when capturing an X-ray image.

The C-arm may adjust a position of the X-ray source through at least one of a longitudinal motion, a lateral motion, a tilting motion, a rotational motion, and a spherical motion, and wherein the control unit controls at least one of the motions of the C-arm to generate a fluoroscopy image while tracking the target based on the position information.

According to one or more exemplary embodiments, an X-ray apparatus includes: a table on which an object is positioned; a data obtaining unit for obtaining position information of a target in the object; and a control unit for moving the table to allow tracking of the target based on the position information when capturing an X-ray image.

The table may adjust a position of the object through at least one of a longitudinal motion, a lateral motion, a tilting motion, and a rotational motion, and wherein the control unit controls at least one of the motions of the table to generate a fluoroscopy image while tracking the target based on the position information.

According to one or more exemplary embodiments, a method of scanning an X-ray includes obtaining position information of a target in an object; and moving at least one of a C-arm for adjusting a position of an X-ray source and a table on which the object is positioned to allow tracking of the target based on the position information when capturing an X-ray image.

The moving of the at least one of the C-arm and table may include: moving at least one of the C-arm and the table to allow the target to be positioned within a predetermined distance from the center of the X-ray image based on the position information.

The moving of the at least one of the C-arm and table may include: moving at least one of the C-arm and the table to allow the target to be positioned in the center of the X-ray image based on the position information.

The moving of the at least one of the C-arm and table may include: moving at least one of the C-arm and the table to allow tracking of at least one of the target and an ROI based on the position information when capturing the X-ray image.

The moving of the at least one of the C-arm and table may include: allowing the target to be positioned within a predetermined distance from the center of the X-ray image based on the position information, recognizing a boundary between the object and the target, and moving at least one of the C-arm and the table to track an ROI based on the boundary.

The C-arm may adjust a position of the X-ray source through at least one of a longitudinal motion, a lateral motion, a tilting motion, a rotational motion, and a spherical motion.

The table may adjust a position of the object through at least one of a longitudinal motion, a lateral motion, a tilting motion, a rotational motion, and a spherical motion.

The obtaining of the position information may include: obtaining the position information of the target based on electrode signals detected from a plurality of electrodes attached to the object.

According to one or more exemplary embodiments, there is provided a non-transitory computer-readable recording medium having recorded thereon a program for executing the method of scanning an X-ray.

### MODE FOR THE INVENTION

This application claims the benefit of Korean Patent Application No. 10-2014-0124630, filed on Sep. 18, 2014, in the Korean Intellectual Property Office, the disclosure of which is incorporated herein in its entirety by reference.

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

Advantages and features of one or more exemplary embodiments and methods of accomplishing the same may be understood more readily by reference to the following detailed description of the embodiments and the accompanying drawings. In this regard, the present embodiments may have different forms and should not be construed as being limited to the descriptions set forth herein. Rather, these embodiments are provided so that this disclosure will be thorough and complete and will fully convey the concept of the present embodiments to one of ordinary skill in the art, and the exemplary embodiments will only be defined by the appended claims.

Hereinafter, the terms used in the specification will be briefly described, and then the exemplary embodiments will be described in detail.

The terms used in this specification are those general terms currently widely used in the art in consideration of functions regarding the exemplary embodiments, but the terms may vary according to the intention of those of ordinary skill in the art, precedents, or new technology in the art. Also, some terms may be arbitrarily selected by the applicant, and in this case, the meaning of the selected terms will be described in detail in the detailed description of the present specification. Thus, the terms used in the specification should be understood not as simple names but based on the meaning of the terms and the overall description of the invention.

Throughout the specification, an “image” may denote multi-dimensional data composed of discrete image elements (for example, pixels in a two-dimensional image and voxels in a three-dimensional image). For example, an image may include medical images of an object acquired by an X-ray, a computed tomography (CT), a magnetic resonance imaging (MRI), an ultrasound wave, and other medical image systems.

Furthermore, in the present specification, an “object” may be a human, an animal, or a part of a human or animal. For example, the object may be an organ (e.g., the liver, the heart, the womb, the brain, a breast, or the abdomen), a blood vessel, or a combination thereof. Furthermore, the “object” may be a phantom. The phantom means a material having a density, an effective atomic number, and a volume that are approximately the same as those of an organism. For example, the phantom may be a spherical phantom having properties similar to the human body.

Furthermore, in the present specification, a “user” may be, but is not limited to, a medical expert, such as a medical doctor, a nurse, a medical laboratory technologist, or a technician who repairs a medical apparatus.

An X-ray apparatus is a medical imaging apparatus that acquires images of internal structures of an object by transmitting an X-ray through the human body. The X-ray apparatus may acquire medical images of an object more simply within a shorter time than other medical imaging apparatuses including an MRI apparatus and a CT apparatus. Therefore, the X-ray apparatus is widely used in simple chest photographing, simple abdomen photographing, simple skeleton photographing, simple nasal sinuses photographing, simple neck soft tissue photographing, and breast photographing.

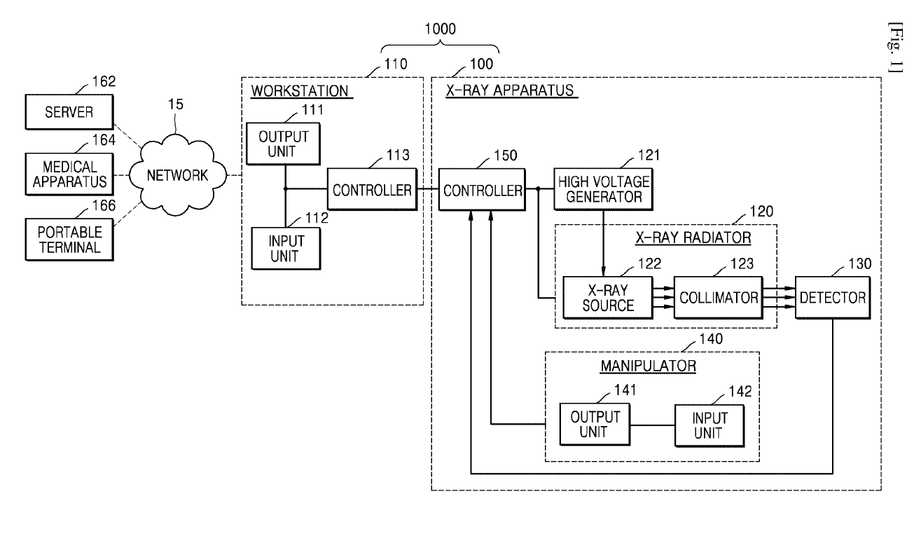

FIG. 1 is a block diagram of an X-ray apparatus 100. The X-ray apparatus 100 shown in FIG. 1 may be a fixed-type X-ray apparatus or a mobile X-ray apparatus.

Referring to FIG. 1, the X-ray apparatus 100 includes a workstation 110, an X-ray irradiation unit 120, a high voltage generator 121, and a detector 130.

The workstation 110 includes an input unit 111 through which a user may input commands for manipulating the X-ray apparatus 100 including an X-ray irradiation, and a control unit 112 controlling overall operations of the X-ray apparatus 100.

The high voltage generator 121 generates a high voltage for generating X-rays, and applies the high voltage to an X-ray source 122.

The X-ray irradiation unit 120 includes the X-ray source 122 receiving the high voltage applied from the high voltage generator 121 to generate and irradiate the X-ray, and a collimator 123 for guiding a path of the X-ray irradiated from the X-ray source 122.

The detector 130 detects an X-ray that is radiated from the X-ray radiator 120 and has been transmitted through an object.

Also, the X-ray apparatus 100 may further include a manipulation unit 140 including a sound output unit 141 outputting sound representing information relating to photographing operation such as the X-ray irradiation under a control of the control unit 112.

The workstation 110, the X-ray irradiation unit 120, the high voltage generator 121, and the detector 130 may be connected to each other via wires or wirelessly. If they are connected to each other wirelessly, a device (not shown) for synchronizing clocks with each other may be further included.

The input unit 111 may include a keyboard, a mouse, a touch screen, a voice recognizer, a fingerprint recognizer, an iris recognizer, and the like well known in the art. The user may input a command for irradiating the X-ray via the input unit 111, and to do this, the input unit 111 may include a switch for inputting the command. The switch may be configured so that an irradiation command for irradiating the X-ray may be input only when the switch is pushed twice.

That is, when the user pushes the switch, a prepare command for performing a pre-heating operation for X-ray irradiation may be input through the switch, and then, when the user pushes the switch once more, the irradiation command for irradiating the X-ray may be substantially input through the switch. When the user manipulates the switch as described above, the input unit 111 generates signals corresponding to the commands input through the switch manipulation, that is, a prepare signal and an irradiation signal, and outputs the generated signals to the high voltage generator 121 generating a high voltage for generating the X-ray.

When the high voltage generator 121 receives the prepare signal output from the input unit 111, the high voltage generator 121 starts a pre-heating operation, and when the pre-heating is finished, the high voltage generator 121 outputs a ready signal to the control unit 121. In addition, the detector 130 also needs to prepare for detecting the X-ray, and thus, when the high voltage generator 121 receives the prepare signal output from the input unit 111, the high voltage generator 121 outputs a prepare signal to the detector 130 at the same time of performing the pre-heating operation, so that the detector 130 may prepare for detecting the X-ray transmitted through the object. The detector 130 prepares for detecting the X-ray when receiving the prepare signal, and when the preparing for the detection is finished, the detector 130 outputs a ready signal to the high voltage generator 121 and the control unit 112.

When the pre-heating operation of the high voltage generator 121 is finished, the detector 130 is ready for the detecting the X-ray, and the irradiation signal is output from the input unit 111 to the high voltage generator 121, the high voltage generator 121 generates and applies the high voltage to the X-ray source 122, and the X-ray source 122 irradiates the X-ray.

When the irradiation signal is output from the input unit 111, the control unit 112 may output a sound output signal to the sound output unit 141 so that the sound output unit 141 outputs predetermined sound and the object may recognize the irradiation of X-ray. Also, the sound output unit 141 may output sound representing other information relating to the photographing, in addition to the X-ray irradiation. In FIG. 1, the sound output unit 141 is included in the manipulation unit 140; however, the exemplary embodiments are not limited thereto, and the sound output unit 140 may be located at a different location from the manipulation unit 140. For example, the sound output unit 141 may be included in the workstation 110, or may be located on a wall surface of an examination room in which the X-ray photographing of the object is performed.

The control unit 112 controls locations of the X-ray irradiation unit 120 and the detector 130, a photographing timing, and photographing conditions according to photographing conditions set by the user.

In more detail, the control unit 112 controls the high voltage generator 121 and the detector 130 according to the command input via the input unit 111 so as to control an irradiation timing of the X-ray, an intensity of the X-ray, and an irradiation region of the X-ray. Also, the control unit 112 adjusts the location of the detector 130 according to a predetermined photographing condition, and controls an operation timing of the detector 130.

In addition, the control unit 112 generates a medical image of the object by using image data transmitted from the detector 130. In detail, the control unit 121 receives the image data from the detector 130, and then, generates the medical image of the object by removing noise in the image data, and adjusting a dynamic range and interleaving of the image data.

The X-ray apparatus 100 shown in FIG. 1 may further include an output unit (not shown) for outputting the medical image generated by the control unit 112. The output unit may output information that is necessary for the user to manipulate the X-ray apparatus 100, for example, a user interface (UI), user information, or object information. The output unit may include a printer, a cathode ray tube (CRT) display, a liquid crystal display (LCD), a plasma display panel (PDP), an organic light emitting diode (OLED) display, a field emission display (FED), a light emitting diode (LED) display, a vacuum fluorescent display (VFD), a digital light processing (DLP) display, a primary flight display (PFD), a three-dimensional (3D) display, a transparent display, and other various output devices well known in the art.

The workstation 110 shown in FIG. 1 may further include a communication unit (not shown) that may be connected to a server 162, a medical apparatus 164, and a portable terminal 166 via a network 150.

The communication unit may be connected to the network 150 via wires or wirelessly to communicate with the external server 162, the external medical apparatus 164, or the external portable terminal 166. The communication unit may transmit or receive data relating to diagnosis of the object via the network 150, and may transmit or receive medical images captured by the other medical apparatus 164, for example, a CT, an MRI, or an X-ray apparatus. Moreover, the communication unit may receive medical history or treatment schedule of an object (e.g., a patient) from the server 162 to diagnose a disease of the object. Also, the communication unit may perform data communication with the portable terminal 166 such as a mobile phone of a doctor or a patient, a personal digital assistant (PDA), or a laptop computer, as well as the server 162 or the medical apparatus 164 in a hospital.

The communication unit may include one or more elements enabling to communicate with external apparatuses, for example, a short distance communication module, a wired communication module, and a wireless communication module.

The short distance communication module is a module for communicating with a device located within a predetermined distance. The short distance communication technology may be wireless local area network (LAN), Wi-Fi, Bluetooth, Zigbee, Wi-Fi Direct (WFD), ultra wideband (UWD), infrared data association (IrDA), Bluetooth low energy (BLE), near field communication (NFC), or the like; however, the exemplary embodiments are not limited thereto.

The wired communication module is a module for communicating by using an electric signal or an optical signal, and the wired communication technology may be wired communication technology using a pair cable, a coaxial cable, or an optical fiber cable, and a wired communication technology that is well known in the art.

The wireless communication module may transmit/receive a wireless signal to/from at least one of a base, an external device, and a server in a mobile communication network. Here, the wireless signal may be a voice call signal, a video call signal, or various types of data according to text/multimedia messages transmission.

The X-ray apparatus 100 shown in FIG. 1 may include a plurality of digital signal processors (DSPs), an ultra-small calculator, and a processing circuit for specialized usage (for example, a high speed analog/digital (A/D) conversion, a high speed Fourier transformation, an array process, etc.).

In addition, the communication between the workstation 110 and the X-ray generator 120, the workstation 110 and the high voltage generator 211, and the workstation 110 and the detector 130 may use a high speed digital interface, such as low voltage differential signalling (LVDS), asynchronous serial communication, such as universal asynchronous receiver transmitter (UART), synchronous serial communication, or a low latency network protocol, such as a controller area network (CAN), and other various communication methods that are well known in the art may be used.)

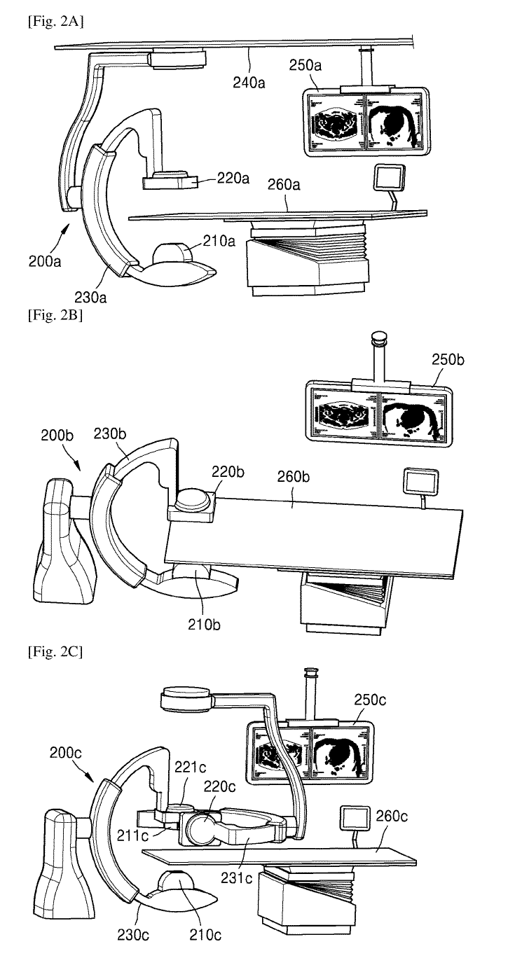

FIGS. 2A through 2C are diagrams of C-arm X-ray apparatuses to which exemplary embodiments are applicable. In more detail, the C-arm X-ray apparatus shown in FIG. 2 is referred to as an interventional X-ray apparatus or an interventional angiography C-arm X-ray apparatus. However, the exemplary embodiments may not be applicable to only the C-arm X-ray apparatus of FIG. 2. For example, the exemplary embodiments may be applicable to a surgical C-arm X-ray apparatus used for surgery. Adjusting of a position of an X-ray source is not only limited to using a C-arm, and the position may be adjusted in other ways. Hereinafter, the meaning of “C-arm X-ray apparatus” includes the interventional X-ray apparatus, the interventional angiography C-arm X-ray apparatus, the surgical C-arm X-ray apparatus, and an X-ray apparatus for adjusting a position of a source.

In more detail, FIG. 2 A shows a ceiling mounted C-arm X-ray apparatus 200a, FIG. 2 B shows a floor mounted C-arm X-ray apparatus 200b, and FIG. 2 C shows a C-arm X-ray apparatus 200c that is a combination of the ceiling mounted C-arm X-ray apparatus and the floor mounted C-arm X-ray apparatus. The C-arm X-ray apparatus 200c of FIG. 2 C may advantageously obtain information two times during the same period of time compared to the ceiling mounted C-arm X-ray apparatus 200a or the floor mounted C-arm X-ray apparatus 200b.

In general, the C-arm X-ray apparatuses 200a, 200b, and 200c may respectively include X-ray sources 210a, 210b, 210c, and 211c, detection units 220a, 220b, 220c, and 221c, C-arms 230a, 230b, 230c, and 231c for respectively adjusting positions of the X-ray sources 210a, 210b, 210c, and 211c and the detection units 220a, 220b, 220c, and 221c, display units 250a, 250b, 250c, and tables 260a, 260b, and 260c on which an object is positioned.

The C-arm X-ray apparatuses 200a, 200b, and 200c of FIG. 2 may be included in or may correspond to the X-ray apparatus 100 of FIG. 1. In more detail, the C-arm X-ray apparatuses 200a, 200b, and 200c, the detection units 220a, 220b, 220c, and 221c, and the display units 250a, 250b, 250c may respectively correspond to an X-ray source 122, the detection unit 130, and the output unit (not shown).

The ceiling mounted C-arm X-ray apparatus 200a of FIG. 2 may further include a guide rail 240a for moving positions of the X-ray source, the detection unit, and the C-arm.

The guide rail 240a is installed in a ceiling of an inspection room in which the C-arm X-ray apparatuses 200a and 200c are disposed. The guide rail 240 may move positions of the X-ray source, the detection unit, and the C-arm by installing a roller (not shown) which is movable along the guide rail 240a. In more detail, longitudinal motion and lateral motion of the C-arm X-ray apparatus may be performed by using the guide rail 240a.

The above-described guide rail 240a may also be installed in the combination C-arm X-ray apparatus 200c of FIG. 2 C.

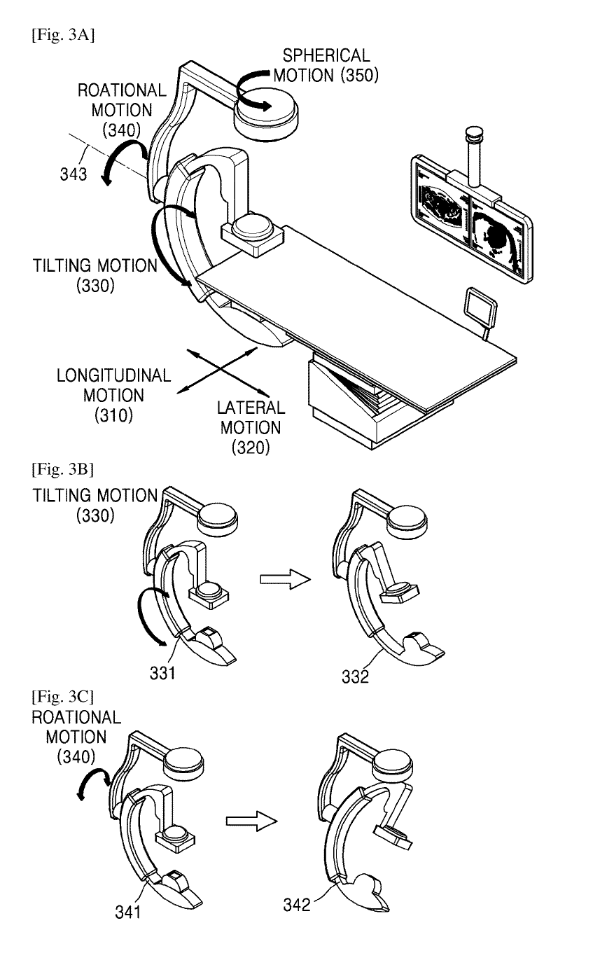

A user may scan the object at various positions or various angles by using the C-arm 230a, 230b, 230c, and 231c and/or tables 260a, 260b, and 260c. For example, the user may scan a region of interest of the object by rotating the C-arm 230a, 230b, 230c, and 231c and/or the tables 260a, 260b, and 260c or moving them up and down left and right to obtain a fluoroscopy image. Thus, the user may more efficiently scan the object by using the C-arm X-ray apparatuses 200a, 200b, and 200c compared to a general fixed type X-ray apparatus. Motions of the C-arm 230a, 230b, 230c, and 231c of the C-arm X-ray apparatuses 200a, 200b, and 200c will be described in detail with reference to FIGS. 3A through 3D. Motions of the tables 260a, 260b, and 260c of the C-arm X-ray apparatuses 200a, 200b, and 200c will be described in detail with reference to FIGS. 3A through 3D.

The C-arm X-ray apparatuses 200a, 200b, and 200c may be useful in medical treatments such as X-ray angiography or a surgical operation. In these medical treatments, an X-ray image of the object needs to be continuously checked during the surgery, and a fluoroscopy image needs to be acquired by continuously irradiating an X-ray to the object.

For example, in the angiography, a guide wire may be installed in a portion of the object to perform X-ray scanning or a thin needle may be used to inject drug to perform X-ray scanning.

As another example, in the surgical operation, when performing surgery by inserting a catheter, a stent, or a needle or the like into the body, the user, for example a doctor, needs to check whether the catheter or the like is normally inserted into a target point of the object. Thus, the user may acquire a fluoroscopy image during treatment, and may conduct the treatment by checking a position of the target such as the catheter by viewing the acquired fluoroscopy image.

The exemplary embodiments may be usefully applied to a case where a conductive material such as the guide wire, the needle, the catheter, or the stent is installed or inserted into the portion of the object and X-ray scanning is performed on the portion of the object by using a C-arm X-ray apparatus. Thus, the exemplary embodiments may enable the X-ray to be exactly irradiated onto the object by exactly determining a position of the object to which the X-ray is irradiated and adjusting a position and/or a direction of at least one of a C-arm and a table. An operation of the X-ray apparatus according to the exemplary embodiments will be described in detail below with reference to FIGS. 3 through 24.

FIG. 3A through FIG. 3D are diagrams for explaining operations of C-arms of an X-ray apparatus according to an exemplary embodiment. FIG. 3A through FIG. 3D illustrate representative motions of the C-arm 230a of the ceiling mounted X-ray apparatus 200a. However, the motions of the C-arm 230a may be applicable to not only the ceiling mounted X-ray apparatus 200a but also the floor mounted C-arm X-ray apparatus 200b, the combination C-arm X-ray apparatus 200c, and other C-arm X-ray apparatuses.

The C-arm 230a may adjust a position of an X-ray source through at least one motion among a longitudinal motion 310, a lateral motion 320, a tilting motion 330, a rotational motion 340, and a spherical motion 350. The C-arm 230a may adjust a position of a detection unit so as to correspond to the position of the X-ray source.

The longitudinal motion 310 is used to move the position of the X-ray source in a longitudinal direction. For example, when a user is to scan a chest of an object from right to left, the user may adjust the position of the X-ray source through the longitudinal motion 310.

The lateral motion 320 is used to move the position of the X-ray source in a lateral direction. For example, when the user is to scan from the chest of the object to the abdomen, the user may adjust the position of the X-ray source through the lateral motion 320.

FIG. 3B is a diagram for explaining the tilting motion 330 of the C-arm 230a. In more detail, FIG. 3B illustrates a C-arm 331 of a reference position before the tilting motion 330 and a C-arm 332 of the reference position after the tilting motion 330. For convenience of illustration, FIG. 3B illustrates the C-arm X-ray apparatus 200a including only the C-arm 230a.

The tilting motion 330 of the C-arm 230a is used to move the position of the X-ray source in a clockwise direction or in a counterclockwise direction. The user may adjust the X-ray source at various positions according to an angle of the tilting motion 330.

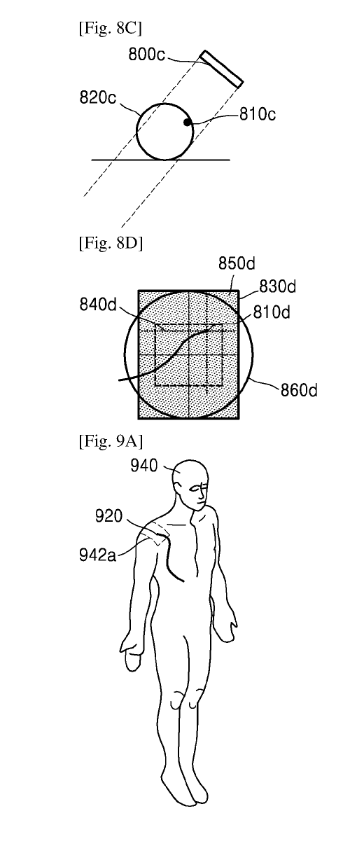

For better understanding, when the C-arm 230a has a semicircular shape, the reference position of the C-arm 230a before the tilting motion 330 may have a left semicircular shape (an angle of the tilting motion 330=0 degrees). That is, the detection unit may be positioned in a direction of 12 o'clock, and the X-ray source may be positioned in a direction of 6 o'clock. The user may adjust the C-arm 230a in a lower semicircular shape by tilting the C-arm 230a by 90 degrees at the reference position in the counterclockwise direction. That is, the detection unit may be positioned in a direction of 9 o'clock, and the X-ray source may be positioned in a direction of 3 o'clock. For example, a left figure of FIG. 3B illustrates the C-arm 331 having the left semicircular shape (an angle of the tilting motion 330=0 degrees) at the reference position before the tilting motion 330, and a right figure illustrates the C-arm 332 after rotating at about 30 degrees in the counterclockwise direction at the reference position. The tilting motion 330 of the X-ray apparatus of the present exemplary embodiment will be described in more detail with reference to FIG. 9.

FIG. 3C is a diagram for explaining the rotational motion 340 of the C-arm 230a. In more detail, FIG. 3C illustrates a C-arm 341 of a reference position before the rotational motion 340 and a C-arm 342 of the reference position after the rotational motion 340. For convenience of illustration, FIG. 3C illustrates the C-arm X-ray apparatus 200a including only the C-arm 230a.

The rotational motion 340 of the C-arm 230a is used to rotate the C-arm 230a by using a center portion of the C-arm 230a as an axis 343. The user may adjust the X-ray source at various positions according to an angle of the rotational motion 340.

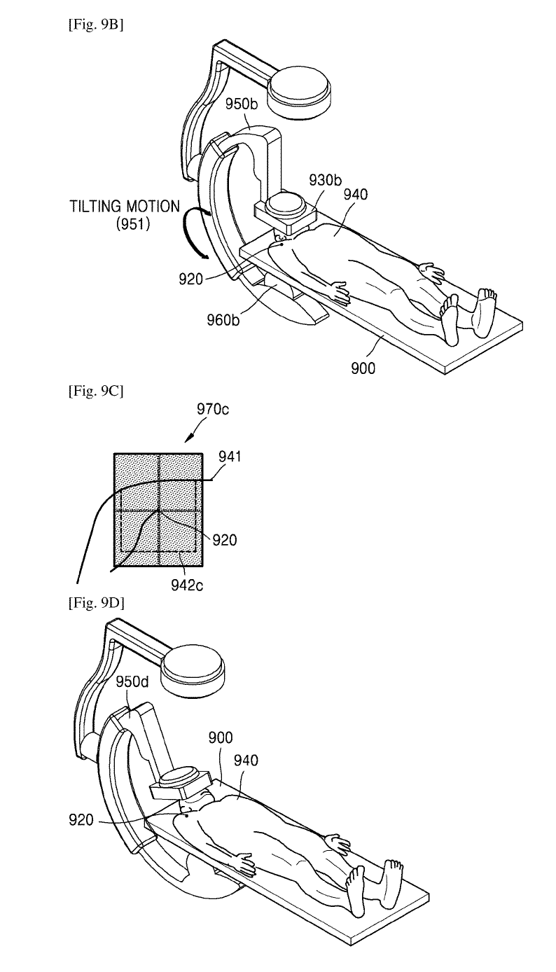

For better understanding, when a table is a plane, an irradiation direction of an X-ray emitted from the X-ray source connected to the C-arm 260a before the rotational motion 340 may be a direction (an angle of the rotational motion 340=0 degrees) perpendicular to the plane. The user may adjust the irradiation direction of the X-ray to be a direction parallel to the plane by rotating the C-arm 230a by 90 degrees at the reference position. As another example, the user may adjust positions of the X-ray source and the detection unit by rotating the C-arm 230a by 180 degrees at the reference position. That is, the user may move the X-ray source at the rear of a back of the object to the above of a chest of the object through the rotational motion 340 by 180 degrees. The rotational motion 340 of the X-ray apparatus of the present exemplary embodiment will be described in more detail with reference to FIG. 10.

FIG. 3D is a diagram for explaining the spherical motion 350 of the C-arm 230a. In more detail, FIG. 3D illustrates a C-arm 351 of a reference position before the spherical motion 350 and a C-arm 352 of the reference position after the spherical motion 350. For convenience of illustration, FIG. 3D illustrates the C-arm X-ray apparatus 200a including only the C-arm 230a.

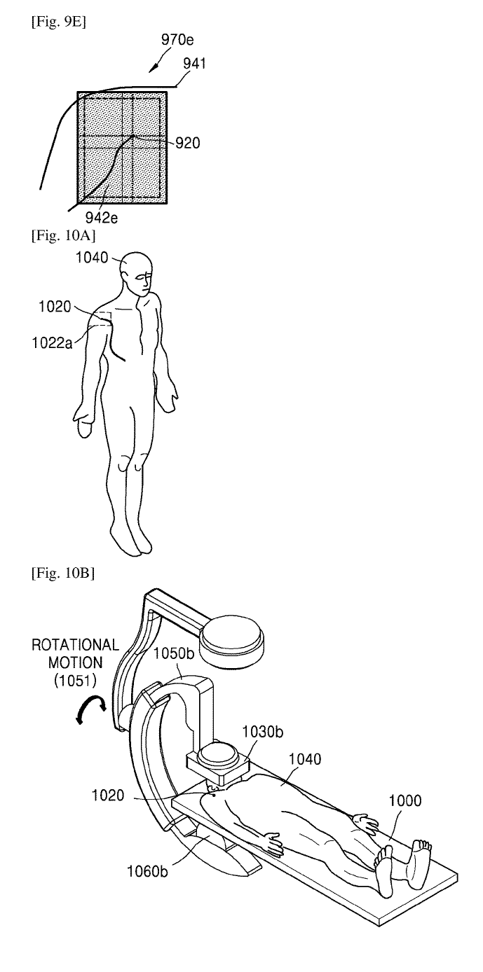

The spherical motion 350 of the C-arm 230a is used to adjust the position of the X-ray source through rotation of an axis connecting the C-arm 230a and a ceiling. The user may adjust a direction of the X-ray source in various ways according to an angle of the spherical motion 350. In particular, when the detection unit has a rectangular or oval shape rather than a square or circular shape, the spherical motion 350 of the C-arm 230a is useful. The spherical motion 350 of the X-ray apparatus of the present exemplary embodiment will be described in more detail with reference to FIG. 11.

The user may obtain an image of various points with respect to a same position of the object through at least one of the tilting motion 330, the rotational motion 340, and the spherical motion 350 of the C-arm 230a. The user may obtain an image with respect to various parts of the object that may not be obtained through the longitudinal motion 310 and the lateral motion 320 through at least one of the tilting motion 330, the rotational motion 340, and the spherical motion 350 of the C-arm 230a.

The motions of the C-arm 230a are not limited to those described above. Additional motions for freely adjusting the position of the X-ray source may be implemented. For example, a motion of raising or lowering the C-arm 230a to the ceiling or the floor may be implemented. Accordingly, the user may expand or reduce the image of the X-ray.

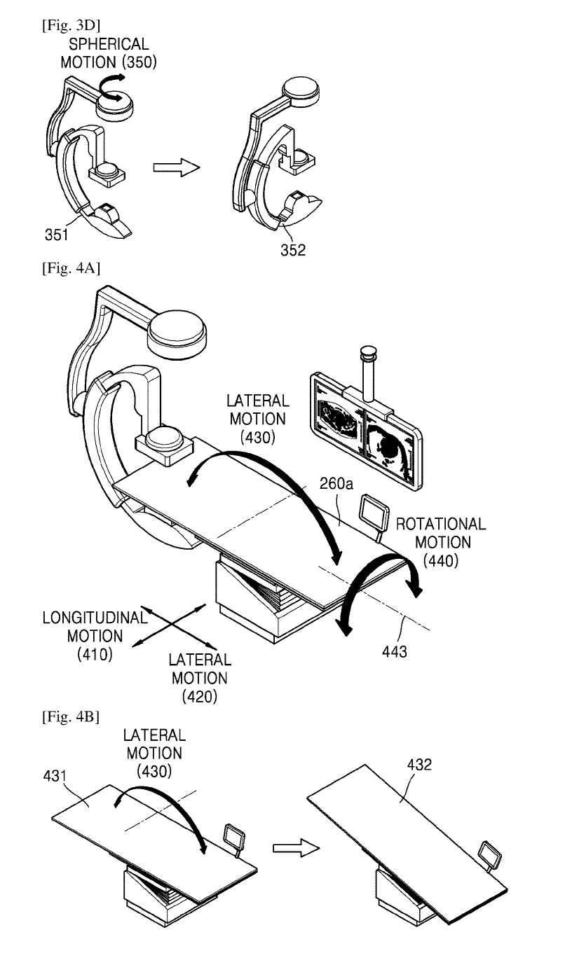

FIGS. 4A through 4C are diagrams for explaining operations of tables of an X-ray apparatus according to an exemplary embodiment. FIGS. 4A through 4C illustrate representative motions of the table 260a of the ceiling mounted C-arm X-ray apparatus 200a. However, the motions of the tables 260a of FIG. 4 may be applicable to not only the ceiling mounted X-ray apparatus 200a but also the floor mounted C-arm X-ray apparatus 200b, the combination C-arm X-ray apparatus 200c, and other C-arm X-ray apparatuses.

An object is positioned on the table 260a. Thus, a user may adjust a position of the object by moving the table 260a and accordingly adjust a part of the object to which an X-ray is irradiated.

In more detail, the table 260a may adjust the position of the object through at least one of a longitudinal motion 410, a lateral motion 420, a tilting motion 430, and a rotational motion 440.

The longitudinal motion 410 of the table 260a is used to move a position of the object in a longitudinal direction. For example, when the user is to scan a chest of the object from right to left, the user may adjust the position of the object through the longitudinal motion 410 of the table 260a. The longitudinal motion 410 of the table 260a in one direction may produce the same effect as that of the longitudinal motion 310 of a C-arm in another direction.

The lateral motion 420 of the table 26a is used to move the position of the object in a lateral direction. For example, when the user is to scan from the chest of the object to the abdomen, the user may adjust the position of the object through the lateral motion 420 of the table 260a. The lateral motion 420 of the table 260a in one direction may produce the same effect as that of the lateral motion 320 of the C-arm in another direction.

FIG. 4B is a diagram for explaining the tilting motion 430 of the table 260a. In more detail, FIG. 4B illustrates the table 260a of a reference position before the tilting motion 430 and the table 260a of the reference position after the tilting motion 430. For convenience of illustration, FIG. 4B illustrates the C-arm X-ray apparatus 200a including only the table 260a.

The tilting motion 430 of the table 260a is used to adjust the position of the object in a clockwise direction or in a counterclockwise direction. In more detail, the user may stand the object up by tilting the table 260a in the clockwise direction or lay the object down by tilting the table 260a in the counterclockwise direction. The user may adjust the object to various positions according to an angle of the tilting motion 430.

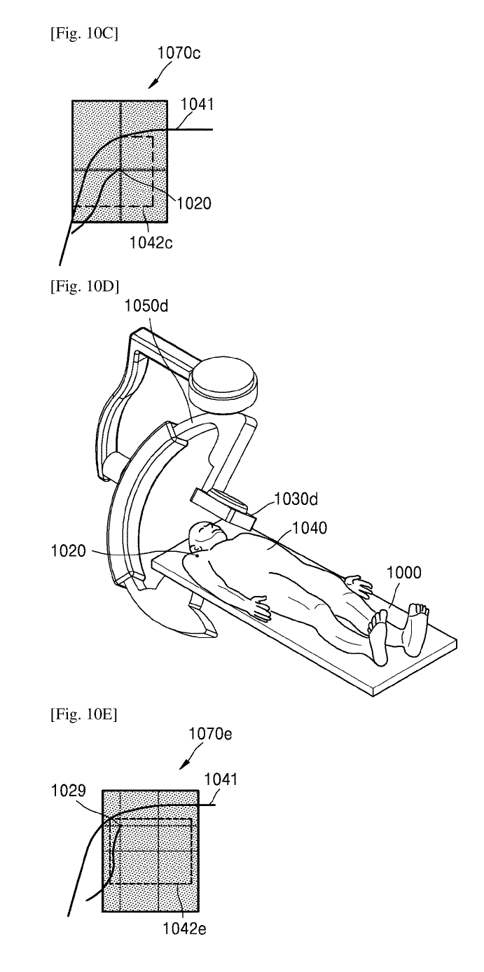

For better understanding, if an upper end of the table 260a at which a head end of the object is positioned is compared to a hour hand, and a lower end of the table 260a at which a minute hand of the object is positioned is compared to a minute hand, the reference position of the table 260a before the tilting motion 430 may have a shape (an angle of the tilting motion 430=0 degrees) of 9:15. The user may adjust the table 260a to a shape of 10:20 by tilting the table 260a by 30 degrees at the reference position in the clockwise direction. That is, the object may be stood up. To the contrary, the user may adjust the table 260a to a shape of 8:10 by tilting the table 260a by 30 degrees at the reference position in the counterclockwise direction. That is, the object may be laid down. The tilting motion 430 of the table 260a in one direction may produce the same effect as that of the tilting motion 330 of the C-arm in another direction. The tilting motion 430 of the X-ray apparatus of the present exemplary embodiment will be described in more detail with reference to FIG. 12.

FIG. 4C is a diagram for explaining the rotational motion 440 of the table 260a. In more detail, FIG. 4C illustrates a table 441 of a reference position before the rotational motion 440 and a table 442 of the reference position after the rotational motion 440. For convenience of illustration, FIG. 4C illustrates the C-arm X-ray apparatus 200a including only the table 260a.

The rotational motion 440 of the table 260a is used to rotate the table 260a by using a center line connecting the head end and a foot end of the table 260a as an axis 443. The user may adjust the object at various positions according to an angle of the rotational motion 440.

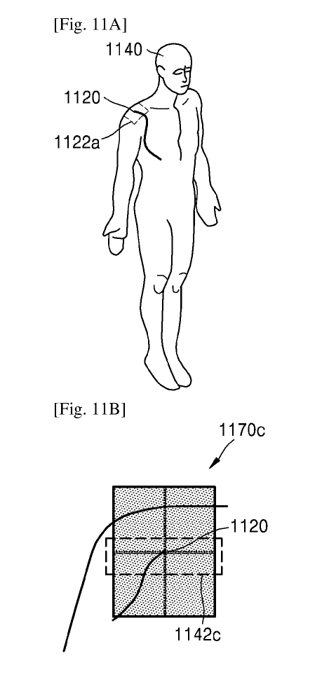

For example, the user may scan the chest of the object from the front on a table (an angle of the rotational motion 440=0 degrees) of a reference position. The user may scan the chest of the object from the side by rotating the table 260a at 30 degrees at the reference position. The rotational motion 440 of the table 260a in one direction may produce the same effect as that of the rotational motion 340 of the C-arm in another direction. The rotational motion 440 of the X-ray apparatus of the present exemplary embodiment will be described in more detail with reference to FIG. 13.

The motions of the table 260a are not limited to those described above. Additional motions for freely adjusting the position of the object may be implemented. For example, a motion of raising or lowering the table 260a to the ceiling or the floor may be implemented. Accordingly, the user may expand or reduce the image of the X-ray.

According to circumstances, the user may more conveniently scan a region of interest of the object by moving a position of an object through a table rather than moving a position of an X-ray source through a C-arm. In particular, when motion of the C-arm can no longer proceed, motion of the table may be useful. To the contrary, if the motion of the table can no longer proceed, the motion of the C-arm may be useful. Thus, the user may efficiently scan the object by freely adjusting the C-arm and the table according to a purpose.

In the related art, if a target goes beyond a fluoroscopy image, the user himself/herself needs to inconveniently adjust the position of the C-arm or the table. Thus, there are problems in that a surgery time increases, and accordingly, a dose of radiation irradiated onto the object unnecessarily increases.

Therefore, one or more exemplary embodiments provide an X-ray apparatus capable of determining position information of a target, tracking the target without manipulation by the user, and, in order to proceed with X-ray scanning, automatically moving at least one of a C-arm and a table. One or more exemplary embodiments may be usefully applied when it monitoring an object that moves or does not move such as a catheter, etc. in real time. For example, one or more exemplary embodiments may be usefully applied to an X-ray apparatus for generating a fluoroscopy image for assisting angiography or surgical treatments as described above.

Hereinafter, according to an exemplary embodiment, an X-ray apparatus capable of moving at least one of a C-arm and a table for generating an X-ray image tracking a target will be described in detail.

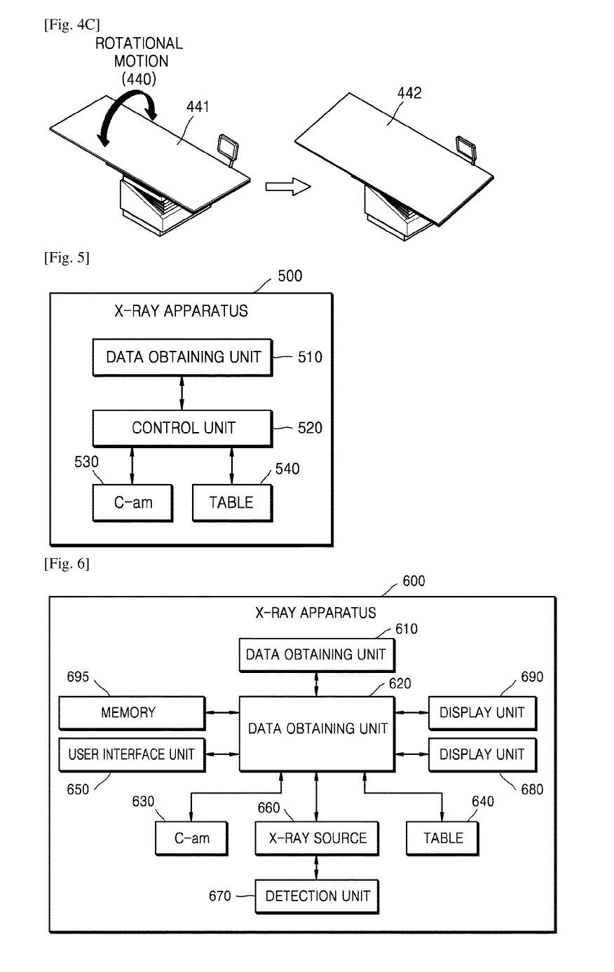

FIG. 5 is a block diagram of an X-ray apparatus 500 according to an exemplary embodiment. The X-ray apparatus 500 according to the present exemplary embodiment may include a data obtaining unit 510 for obtaining position information of a C-arm 530 for adjusting a position of an X-ray source, a table 540 on which an object is positioned, and a target included in the object, and a control unit 520 for moving at least one of the C-arm 530 and the table 540 to allow tracking of the target when capturing an X-ray image.

The X-ray apparatus 500 according to the present exemplary embodiment may be included in or may correspond to the X-ray apparatus 100 of FIG. 1. In more detail, the data obtaining unit 510 and the control unit 520 of FIG. 5 may be included in a workstation 10 of FIG. 1. Alternatively, the control unit 520 of FIG. 5 may be included in or may correspond to the control unit 112 of FIG. 1. Thus, redundant descriptions between FIGS. 5 and 1 are omitted.

The C-arm 530 and the table 540 of FIG. 5 may respectively correspond to the C-arm 230a, 230b, and 230c and the table 260a, 260b, and 260c of FIG. 2. Thus, the C-arm 530 of the X-ray apparatus 500 of the present exemplary embodiment may adjust a position of the X-ray source through at least one of the longitudinal motion 310, the lateral motion 320, the tilting motion 330, the rotational motion 340, and spherical motion 350 described with reference to FIG. 3. The table 540 of the X-ray apparatus 500 of the present exemplary embodiment may adjust a position of the object through at least one of the longitudinal motion 410, the lateral motion 420, the tilting motion 430, and the rotational motion 440 described with reference to FIGS. 4A through 4C. Thus, redundant descriptions between FIGS. 5, 3, and 4 are omitted.

The data obtaining unit 510 of the present exemplary embodiment may obtain the position information of the target included in the object. The target is to be scanned by the user and may be included in the object. The target may be all electrical conductive materials that may be inserted into the object. For example, the target may be a catheter inserted into the object to be used in angiography.

The data obtaining unit 510 may obtain the position information of the target by using various methods. For example, the data obtaining unit 510 may perform image tracking of the target appearing in the X-ray image to obtain position information of the target. That is, the target may be tracked by using image processing performed on the X-ray image. For example, the data obtaining unit 510 may obtain the position information of the target by comparing commonness and/or differences between a plurality of frames.

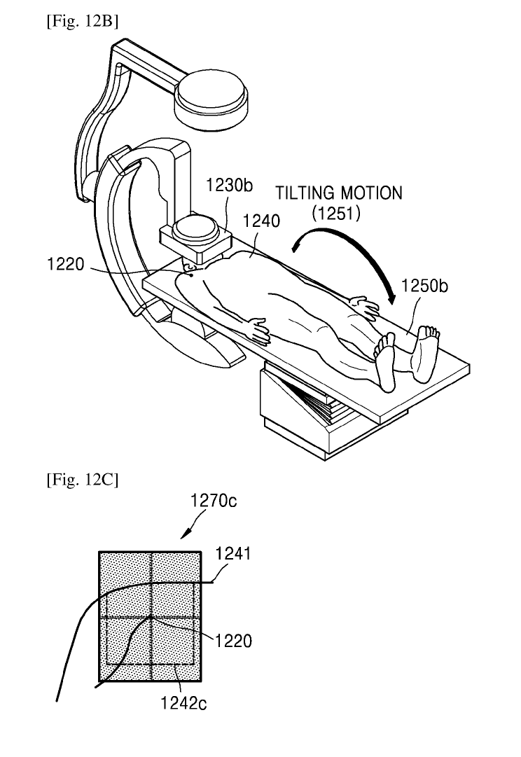

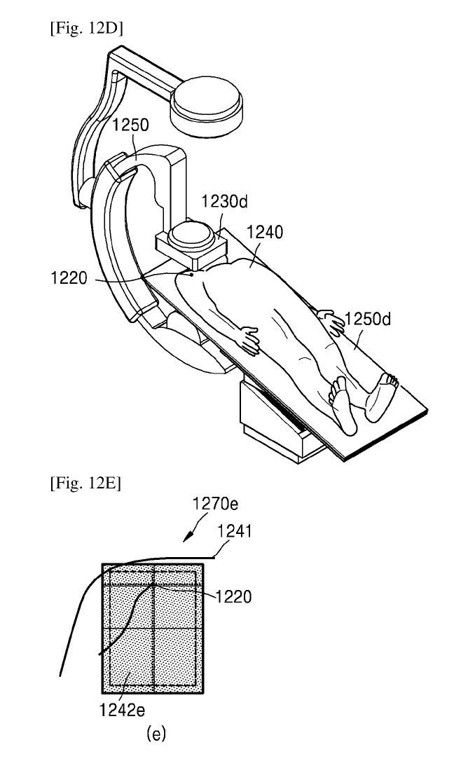

As another example, the data obtaining unit 510 may obtain the position information of the target based on electrode signals detected in a plurality of electrodes attached to the object. In this regard, an operation of the data obtaining unit 510 will be described in detail with reference to FIGS. 16 and 17.

However, the method in which the data obtaining unit 510 obtains the position information of the target is not limited to that described above. For example, the data obtaining unit 510 may obtain the position information of the target through eye-tracking. In this regard, eye-tracking is a method of tracking the target by recognizing a point at which the user stares.

The control unit 520 according to the present exemplary embodiment may move at least one of the C-arm 530 and the table 540 to allow tracking of the target based on the position information of the target when capturing the X-ray image. That is, the control unit 520 may move at least one of the C-arm 530 and the table 540 such that the target may be continuously included in the X-ray image.

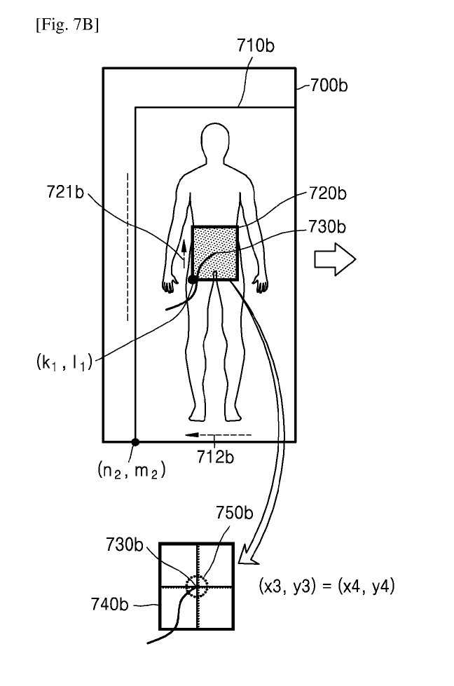

For example, the control unit 520 may move at least one of the C-arm 530 and the table 540 to generate a fluoroscopy image used to track the target. Thus, the user may efficiently monitor a medical surgery such as angiography or surgical treatments. The X-ray image of the present exemplary embodiment may include the fluoroscopy image. An operation of the control unit 520 will be described in detail with reference to FIGS. 7 through 24.

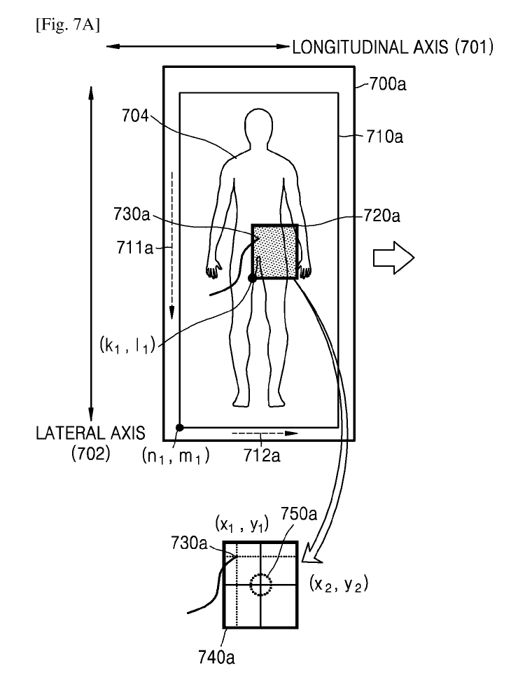

FIG. 6 is a block diagram of an X-ray apparatus 600 according to another exemplary embodiment. The X-ray apparatus 600 of FIG. 6 may further include one of a user interface unit 650, an X-ray source 660, a detection unit 670, a display unit 680, a communication unit 690, and a memory 695, compared to the X-ray apparatus 500 of FIG. 5. Other elements may correspond to those of FIG. 5. Thus, redundant descriptions between FIGS. 6 and 5 are omitted.

In more detail, the X-ray apparatus 600 of the present exemplary embodiment may further include the X-ray source 660 that emits an X-ray to an object and the detection unit 670 that detects the X-ray passing through the object. The C-arm 630 may connect the X-ray source 660 and the detection unit 670 and adjust positions of the X-ray source 660 and the detection unit 670.

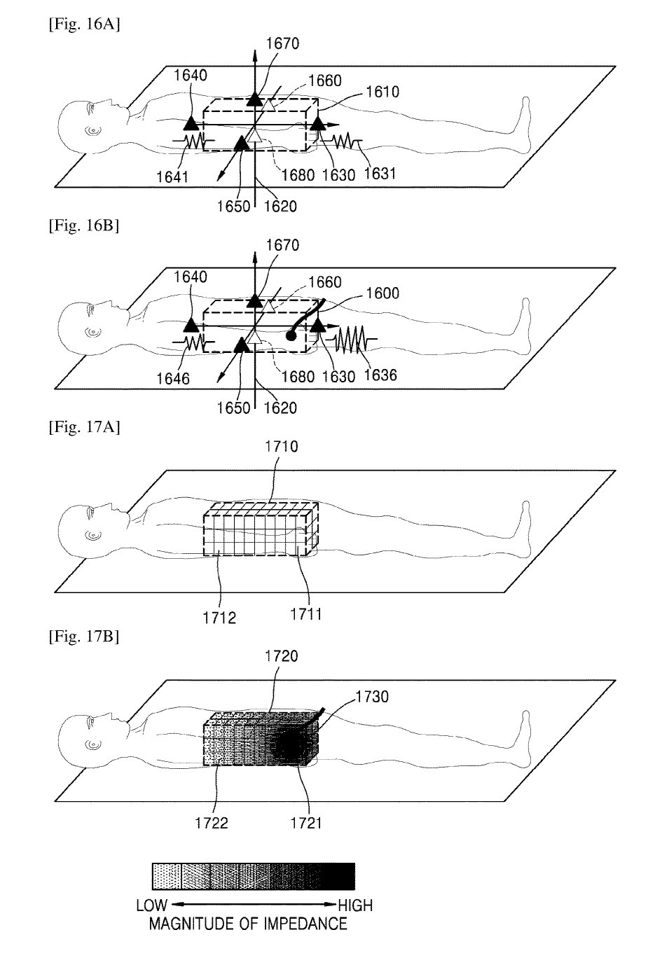

The X-ray source 660, the detection unit 670, and the display unit 680 of FIG. 6 may respectively correspond to the X-ray source 122, the detection unit 130, and the output unit (not shown) of FIG. 1. Alternatively, the X-ray source 660, the detection unit 670, and the display unit 680 of FIG. 6 may respectively correspond to the X-ray sources 210a, 210b, 210c, and 211c, the detection units 220a, 220b, 220c, and 221c, and the display units 250, 250b, and 250c of FIG. 21. Thus, redundant descriptions between FIGS. 6, 1, and 2 are omitted.

The communication unit 690 of the present exemplary embodiment may transmit and receive predetermined data to and from an external apparatus over a wired and/or wireless network. For example, the communication unit 690 may correspond to the communication unit (not shown) of FIG. 1, and may transmit and receive predetermined data to and from the external server 162, the medical apparatus 164, and the portable terminal 166.

The memory 695 of the present exemplary embodiment may store various pieces of data related to an X-ray image. For example, the memory 695 may store at least one of a scanned X-ray image, position information of a target, and current position information of a C-arm and a table.

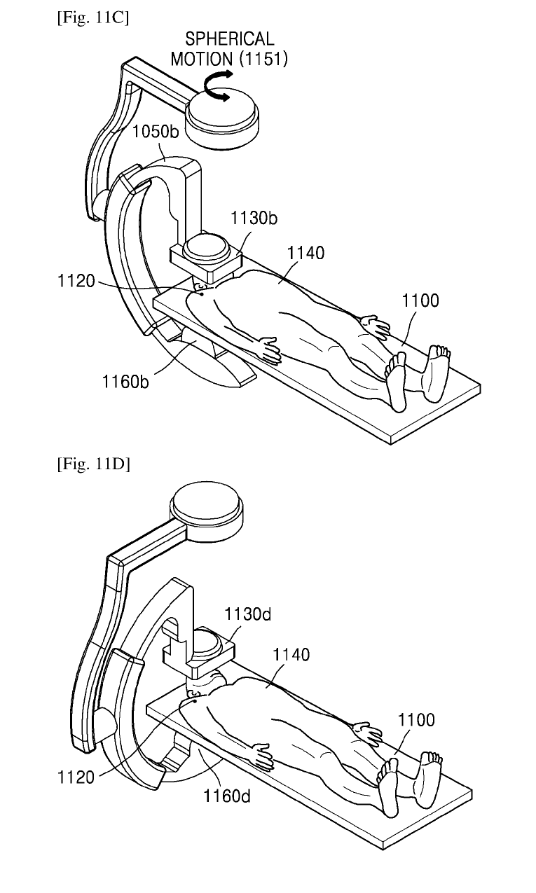

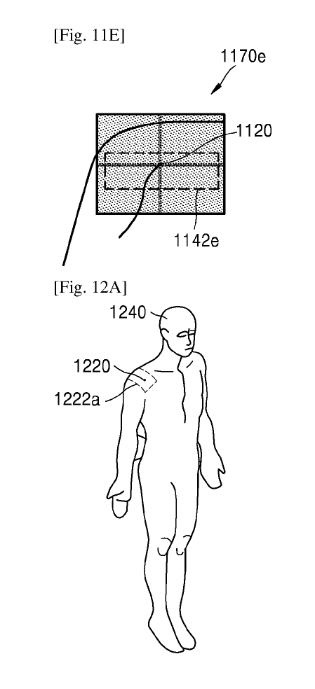

The user interface unit 650 of the present exemplary embodiment may receive a user input. The control unit 620 may move at least one of the C-arm 630 and a table 640 based on the received user input and the position information of the target. The user interface unit 650 will be described in detail with reference to FIGS. 14 and 15.

The user interface unit 650 may be formed as a touch pad. In more detail, the user interface unit 650 may include a touch pad (not shown) combined with a display panel (not shown) included in the display unit 680. The display unit 680 displays a user interface screen on the display panel. If a user touches a predetermined point on the user interface screen in order to input a predetermined command, the touch pad detects the touched point to recognize the predetermined command input by the user.

In more detail, when the user interface unit 650 is formed as the touch pad, if the user touches the predetermined point on the user interface screen, the user interface unit 650 detects the touched point. The user interface unit 650 may transmit detected information to the control unit 620. Then, the control unit 620 may recognize a user request or command corresponding to the detected information and perform the recognized request or command. An operation of the user interface unit 650 will be described in detail with reference to FIGS. 14 and 15.

The control unit 620, the display unit 680, and the user interface unit 650 may be connected to each other by wire or wirelessly and may transmit and receive predetermined data therebetween.

FIGS. 7A through 7C are diagrams for explaining operations of an X-ray apparatus according to an exemplary embodiment. In more detail, FIG. 7 is a diagram for explaining the operations of the X-ray apparatus for generating a fluoroscopy image to assist angiography to which the one or more exemplary embodiments are applied. FIG. 7A through FIG. 7C illustrate the operations over time. In FIG. 7, catheters 730a, 730b, and 730c that are targets are inserted into an object 740 and move according to an intention of a user or an objective. Thus, the X-ray apparatus of the present exemplary embodiment may track the targets through longitudinal motion and lateral motion of a C-arm and longitudinal motion and lateral motion of a table to generate the fluoroscopy image.

To make longitudinal and lateral directions of FIG. 7, respectively, identical to longitudinal and lateral directions of FIGS. 3 and 4, a longitudinal direction 701 of the C-arm and the table of FIG. 7 means from a left end direction of the object to a right end direction or an opposite direction. A lateral direction 702 of the C-arm and the table of FIG. 7 means from a head end direction of the object to a foot end direction or an opposite direction.

The table of the present exemplary embodiment may include external tables 700a, 700b, and 700c and internal tables 710a, 710b, and 710c. The longitudinal motion and the lateral motion of the table may be longitudinal motion and lateral motion of the internal tables 710a, 710b, and 710c that move in the external tables 700a, 700b, and 700c. That is, the external tables 700a, 700b, and 700c may set a range or limitation of the longitudinal motion and the lateral motion of the table. The longitudinal motion and the lateral motion of the table mean motions of the internal tables 710a, 710b, and 710c that move in the external tables 700a, 700b, and 700c below. However, in rotational motion and tilting motion of the table described above, the external tables 700a, 700b, and 700c and the internal tables 710a, 710b, and 710c may also rotate or tilt.

For convenience of description, FIG. 7 illustrates the X-ray apparatus only including detection units 720a, 720b, and 720c and the tables 700a, 700b, and 700c at a point in which the object is viewed from above.

A control unit of the X-ray apparatus of the present exemplary embodiment may move at least one of the C-arm and the table to allow tracking of the targets 730a, 730b, and 730c when capturing an X-ray image. For example, when the targets 730a, 730b, and 730c are moved to completely go beyond X-ray images 740a and 740b, the control unit may allow the targets 730a, 730b, and 730c to be included in the X-ray images 740a and 740b by moving at least one of the C-arm and the table.

Alternatively, the control unit may move at least one of the C-arm and the table in such a manner that allows the targets 730a, 730b, and 730c to be positioned within a predetermined distance from the center of the X-ray images 740a and 740b based on position information of the targets 730a, 730b, and 730c. In more detail, when predetermined circles having a diameter r with respect to the center of the X-ray images 740a and 740b are set as boundaries 750a and 750b, and the targets 730a, 730b, and 730c go beyond the boundaries 750a and 750b, the control unit may allow the targets 730a, 730b, and 730c to be included in the boundaries 750a and 750b again by moving at least one of the C-arm and the table. For example, when the targets 730a, 730b, and 730c are within the boundaries 750a and 750b, and the targets 730a, 730b, and 730c go beyond the boundaries 750a and 750b by not controlling the C-arm and the table, the control unit may allow the targets 730a, 730b, and 730c to be positioned in the center of the X-ray images 740a and 740b by moving at least one of the C-arm and the table. The user may set ranges or shapes of the boundaries 750a and 750b in various ways according to objectives.

Alternatively, the control unit may move at least one of the C-arm and the table in such a manner as to allow the targets 730a, 730b, and 730c to be positioned in the center of the X-ray images 740a and 740b constantly based on the position information of the targets 730a, 730b, and 730c.

A number of times the control unit moves the C-arm or the table may be changed in various ways according to a predetermined range (all the X-ray images 740a and 750a, within a predetermined distance from the center of the X-ray images 740a and 750a, and the center of the X-ray images 740a and 750a) by which the targets 730a, 730b, and 730c may be positioned within the X-ray images 740a and 750a. That is, the smaller the predetermined range by which the targets 730a, 730b, and 730c may be positioned within the X-ray images 740a and 750a, the more frequently the control unit may move the C-arm or the table. Thus, the user may set the predetermined range by which the targets 730a, 730b, and 730c may be positioned within the X-ray images 740a and 750a in accordance with a user's intention of an objective of surgery. The user may change the predetermined range during the surgery.

Hereinafter, examples in which the control unit moves at least one of the C-arm and the table in such a manner as to allow the targets 730a, 730b, and 730c to be positioned within a predetermined distance r from the center of the X-ray images 740a and 750a will be described in detail with reference to FIG. 7.

Referring to FIG. 7, the X-ray apparatus of the present exemplary embodiment may set coordinates for each of the targets 730a, 730b, and 730c, the table, and the C-arm.

For example, referring to FIG. 7A, the data obtaining unit 610 of the present exemplary embodiment may set target coordinates (x1, y1) indicating position information of the target 730a on a coordinate system with respect to the X-ray image 740a. The data obtaining unit 610 of the present exemplary embodiment may set table coordinates (n1, m1) indicating position information of the target 710a on a coordinate system with respect to the external table 700a. The data obtaining unit 610 of the present exemplary embodiment may set C-arm coordinates (k1, l1) indicating position information of the C-arm with respect to a guide rail.

The X-ray apparatus of the present exemplary embodiment may control longitudinal motion and lateral motion of the C-arm and the table based on the set coordinates and Equation 1 below.

f(Δx,Δy)=t(Δn,Δm)+c(Δk,Δl) [Equation 1]

In Equation 1, f(Δx, Δy) is a function with respect to the position information of the target 730a. In more detail, f(Δx, Δy) may be a difference between the center (x2, y2) of the X-ray image 740a and the position coordinates (x1, y1) of the current target 730a on a coordinate system with respect to the X-ray image 740a, Δx may be a longitudinal difference, and Δy may be a lateral difference. For example, f(Δx, Δy) may be a difference between the position coordinates (x1, y1) of the target 730a having pixels of the X-ray image 740a as units and the center (x2, y2) of the X-ray image 740a.

In Equation 1, t(Δn, Δm) is a movement function of the table. That is, t(Δn, Δm) may be the function with respect to a movement distance of the table that needs to be moved in such a manner that the control unit may allow the target 730a to be positioned in the center of the X-ray image 740a. In more detail, the control unit 620 may move the table in a longitudinal direction by a displacement Δn through longitudinal motion of the table and may move the table in a lateral direction by a displacement Δm through lateral motion of the table. Thus, (Δn, Δm) may be a difference between positions (n2, m2) of the table after longitudinal motion and lateral motion and positions (n1, m1) of the table before longitudinal motion and lateral motion.

In Equation 1, c(Δk, Δl) may be a movement function of the C-arm. That is, c(Δk, Δl) may be a function with respect to a displacement of the C-arm that needs to be moved in such a manner that the control unit may allow the target 730a to be positioned in the center of the X-ray image 740a. In more detail, the control unit 620 may move the C-arm in a longitudinal direction by a displacement Δk through longitudinal motion of the C-arm and may move the C-arm in a lateral direction by a displacement Δl through lateral motion of the C-arm. Thus, (Δk, Δl) may be a difference between positions (k2, l2) of the C-arm after longitudinal motion and lateral motion and positions (k1, l1) of the C-arm before longitudinal motion and lateral motion.

A ratio of a size of an object on an X-ray image and a size of an actual object may not be 1:1. That is, the X-ray image may be smaller or larger than the actual object according to a scanning environment or an image processing method. For example, the farther the C-arm is positioned from the object, the smaller the ratio of the size of the X-ray image with respect to the size of the actual object. An X-ray image obtained in the same environment may be enlarged or reduced according to the image processing method, and accordingly, the ratio of the size of the X-ray image with respect to the size of the actual object may be increased or reduced.

Therefore, it may be necessary to standardize the movement function t(Δn, Δm) of the table and the movement function c(Δk, Δl) of the C-arm with respect to a size of the X-ray image 740a so that the control unit may allow the target 730a to be positioned in the center of the X-ray image 740a. That is, t(Δn, Δm) and c(Δk, Δl) of Equation 1 may be respectively the movement function of the table and the movement function of the C-arm that are standardized with respect to the size of the X-ray image 740a. For example, when the size of the actual object: the size of the object on the X-ray image 740a=2:1, the control unit may move the table by sizes of Δx, Δy two times to allow the target 730a to be positioned in the center of the X-ray image 740a.

As described above, the X-ray apparatus of the present exemplary embodiment may move at least one of the C-arm and the table in such a manner that the control unit may allow the target 730a to be positioned in the center of the X-ray image 740a based on the position information of the target 730a. In more detail, when the target 730a is within the predetermined distance r from the center of the X-ray image 740a, the control unit does not control the C-arm and the table. However, when the target 730a goes beyond the boundary 750a, the control unit may move at least one of the C-arm and the table to allow the target 730a to be positioned in the center of the X-ray image 740a. For example, the control unit may move at least one of the C-arm and the table according to a condition that may be expressed as Equation 2 below.

\sqrt{Δx²+Δy²}>r [Equation 2]

The control unit may move only the C-arm and only the table to allow the target 730a to be positioned in the center of the X-ray image 740a. The C-arm and the table may move together. In this case, the C-arm may be moved first or the table may be moved first. The user may set an object to be moved or an order according to a scanning environment, and may also set a movement distance ratio for setting how much to move the C-arm and the table. In this regard, operations will be described in detail with reference to FIGS. 14 and 15.

An example of Equation 1, to which the movement function of the table and the movement function of the C-arm that are standardized with respect to the size of the X-ray image 740a described above and the movement distance ratio are reflected, is Equation 3 as shown below.

$$[ \begin{matrix}Δ x \\ Δ y\end{matrix} ] = [ \begin{matrix}{S}_{x} & 0 \\ 0 & {S}_{y}\end{matrix} ] \cdot [ \begin{matrix}Δ n + Δ k \\ Δ m + Δ l\end{matrix} ] \\ [ \begin{matrix}{S}_{x} & 0 \\ 0 & {S}_{y}\end{matrix} ] \cdot [ \begin{matrix}Δ n \\ Δ m\end{matrix} ] = [ \begin{matrix}{R}_{x} \cdot Δ x \\ {R}_{y} \cdot Δ y\end{matrix} ] \\ [ \begin{matrix}{S}_{x} & 0 \\ 0 & {S}_{y}\end{matrix} ] \cdot [ \begin{matrix}Δ k \\ Δ l\end{matrix} ] = [ \begin{matrix}( 1 - {R}_{x} ) \cdot Δ x \\ ( 1 - {R}_{y} ) \cdot Δ y\end{matrix} ] \qquad [ Equation 3 ]$$

Sx and Sy of Equation 3 may be parameters for standardizing the movement function t(Δn, Δm) of the table and the movement function c(Δk, Δl) of the C-arm with respect to the size of the X-ray image 740a. Sx and Sy may be respectively longitudinal and lateral standardization parameters set with respect to the size of the X-ray image 740a. Rx and Ry of Equation 3 have a value between 0 and 1 as the movement distance ratio between the table and the C-arm. In more detail, Rx is a movement distance ratio of longitudinal motion, and Ry is a movement distance ratio of lateral motion, between the table and the C-arm. The higher the Rx and Ry, the more the table is moved and the less the C-arm is moved by the control unit to allow the target 730a to be positioned in the center of the X-ray image 740a. For example, when Rx=Ry=0.8, a movement distance of the table may be four times a movement distance of the C-arm.

FIG. 7A illustrates an initial scanning status of the X-ray apparatus after the target 730a is inserted into the object 740. The X-ray image 740a at the bottom of FIG. 7A is an enlarged view of the X-ray image 740a obtained from the detection unit 720a of FIG. 7A. In the initial scanning status, the user may directly set a position of the C-arm or the table to allow the target 730a to be included in the X-ray image 740a.

According to an exemplary embodiment, if the target 730a is included in the X-ray image 740a, the data obtaining unit may obtain position information of the target 730a within the X-ray image 740a. For example, the data obtaining unit may set initial position information of the target 730a as the coordinates (x1, y1) on the coordinate system with respect to the X-ray image 740a. The data obtaining unit may set initial position information of the table as the coordinates (n1, m1) on the coordinate system with respect to the external table 700a. The data obtaining unit may set initial position information of the C-arm as the coordinates (k1, l1) on the coordinate system with respect to the guide rail.

Referring to FIG. 7A, even if the user allows the target 730a to be included in the X-ray image 740a in the initial scanning status, the current target 730a goes beyond the boundary 750a set as a predetermined distance from the center (x2, y2) of the X-ray image 740a. In this regard, the X-ray apparatus may allow the target 730a to be positioned in the center (x2, y2) of the X-ray image 740a by moving only the table based on a setting of the user or a default setting. That is, it may be set as Rx=Ry=1 of Equation 3. In more detail, the control unit moves the table to a position of (n2, m2) by controlling longitudinal motion 712a and lateral motion 711a of the table based on Equation 4. As a result, the target 730a may be positioned in the center (x2, y2) of the X-ray image 740a.

$$[ \begin{matrix}x 2 - x 1 \\ y 2 - y 1\end{matrix} ] = [ \begin{matrix}{S}_{x} & 0 \\ 0 & {S}_{y}\end{matrix} ] \cdot [ \begin{matrix}n 2 - n 1 \\ m 2 - m 1\end{matrix} ] \\ [ \begin{matrix}{S}_{x} & 0 \\ 0 & {S}_{y}\end{matrix} ] \cdot [ \begin{matrix}n 2 - n 1 \\ m 2 - m 1\end{matrix} ] = [ \begin{matrix}x 2 - x 1 \\ y 2 - y 1\end{matrix} ] \qquad [ Equation 4 ]$$

FIG. 7B illustrates a scanning status after the longitudinal motion 712a and lateral motion 711a of the table 710b based on Equation 4. The X-ray image 740b at the bottom of FIG. 7B is an enlarged view of the X-ray image 740b obtained from the detection unit 720b of FIG. 7B. Position information (x3, y3) of the current target 730b is identical to a center (x4, y4) of the X-ray image 740b.

When the target 730b is positioned within a predetermined distance 750b from the center (x4, y4) of the X-ray image 740b of FIG. 7B, the control unit 620 does not control the C-arm 630 or the table 640. However, when the target 730b goes beyond the boundary 750b, the control unit 620 moves the C-arm 630 or the table 640 again to allow tracking of the target 730b when capturing the X-ray image 740b.

FIG. 7C illustrates an X-ray scanning status after moving the C-arm and the table when the target 730b goes beyond the boundary 750b in a status of FIG. 7B. In this regard, the X-ray apparatus may set Rx=1, Ry=0 based on a user setting or a default setting. In more detail, the control unit moves the table to a position (n3, m3) by controlling longitudinal motion 712b of the table and moves the C-arm to the position (k2, l2) by controlling the lateral motion 721b of the C-arm. As a result, the target may be positioned in the center of the X-ray image.

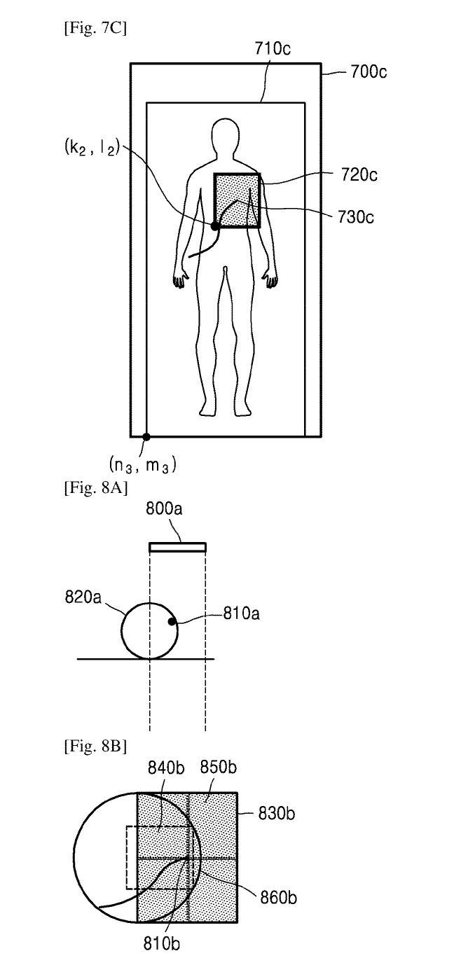

FIGS. 8A through 8D are diagrams for explaining operations of an X-ray apparatus according to another exemplary embodiment. FIGS. 8A through 8D are diagrams for explaining the X-ray apparatus that tracks a region of interest according to an exemplary embodiment. For convenience of description, FIGS. 8A through 8D illustrate objects 820a and 820c in the form of a sphere.

FIG. 8A illustrates a scanning environment when a C-arm is at a reference position. FIG. 8C illustrates the scanning environment after a control unit moves the C-arm to track regions of interest 840b and 840d. FIG. 8B and FIG. 8D illustrate X-ray images 830b and 830d obtained from detection units 800a and 800c of FIG. 8A and FIG. 8C, respectively.

The control unit of the present exemplary embodiment may move at least one of the C-arm and a table to allow tracking of at least one of targets 810b and 810d or the regions of interest 840b and 840d based on position information of the targets when capturing the X-ray images 830b and 830d. That is, the control unit may move at least one of the C-arm and the table to allow tracking of a target, a region of interest, the target included in the region of interest, or the region of interest included in the target when capturing an X-ray image.

In this regard, the regions of interest 840b, 840d, and 840f of an object mean a region of the object that is to be scanned by a user. Thus, in general, the regions of interest 840b, 840d, and 840f of the object do not include empty spaces 850b, 850d, and 850f of the table. The regions of interest 840b, 840d, and 840f are used to observe targets and may include the targets 810a, 810b, 810c, and 810d. The user may set the regions of interest 840b, 840d, and 840f in various ways according to an object of surgery. For example, the regions of interest 840b, 840d, and 840f of FIG. 8B and FIG. 8D may be regions inside dotted lines including targets.

After the control unit of the present exemplary embodiment allows the target to be positioned in the center of the X-ray image by moving the C-arm or the table, the region of interest of the object may not be positioned in the center of the X-ray image.

For example, when the target 810a is positioned in a side end portion of the object 820a and an X-ray irradiation direction is perpendicular to the table as shown in FIG. 8A, the region of interest 840b of the object may be tilted to one side on the X-ray image 830b as shown in FIG. 8B. In this case, the empty space 850b of the table may generate many inefficient images relatively included in the X-ray image 830b. That is, the user may more efficiently conduct surgery when referring to the X-ray image tracking the region of interest rather than the target. Thus, the control unit of the X-ray apparatus of the present exemplary embodiment may move at least one of the C-arm and the table to track the regions of interest 840b, 840d, and 840e.

For example, when a boundary 860b of the object and the table is included in the X-ray image 830b as shown in FIG. 8B, the control unit may select the region of interest 840b as an object to be tracked. Alternatively, when the boundary 860b is present within a predetermined distance from the center of the X-ray image 830b, the control unit may select the region of interest 840b as the object to be tracked.

In more detail, the control unit may recognize the boundary 860b of the object and the table by allowing the targets 810c and 810d to be positioned within the predetermined distance from the center of the X-ray image based on position information of the targets 810a and 810b. That is, the control unit may track the target first and check if the boundary 860b is present around the target. The control unit may recognize the boundary 860b of the empty space 850b of the object and the table through high pass filtering image processing. In addition, the control unit may recognize the boundary 860b through various image processing methods.

As shown in FIG. 8C and FIG. 8D, the control unit may move at least one of the C-arm and the table to track the region of interest 840d of the object based on the recognized boundary 860b and position information of the target 810d.

For example, in FIG. 8C, the control unit may tilt the C-arm by about 45 degrees in a clockwise direction in order to allow the region of interest 840d to be positioned in the center of the X-ray image 830d. In this regard, the control unit may select tilting motion or rotational motion according to an arrangement form of the C-arm.

As a result, at least one of the target 810d and the region of interest 840d of the object may be positioned in the center of the X-ray image 830d. As such, the user may more efficiently monitor the surgery based on the X-ray image 830d of FIG. 8D rather than the X-ray image 830b of FIG. 8B.

The control unit may track the region of interest 840d by using various methods. As described above, the control unit may move at least one of the C-arm and the table to allow the region of interest of the object to be positioned in the center of the X-ray image. As another example, the control unit may move at least one of the C-arm and the table to allow the region of interest of the object to be included in the X-ray image or to allow more than a predetermined portion of the region of interest to be included in the X-ray image. As another example, the control unit may move at least one of the C-arm and the table in proportion to a ratio of an area of the empty space of the table that occupies in the X-ray image. That is, the greater the area of empty space of the table that occupies the X-ray image, the smaller the control unit may reduce the area of empty space that occupies the X-ray image by further tilting or rotating at least one of the C-arm and the table.

In general, when the X-ray image tracks the region of interest, the target may not be positioned in the center of the X-ray image. However, the target and the region of interest may be positioned in the center of the X-ray image according to a position of the target, a shape of the region of interest, etc.

In general, when the boundary 860b of the object and the table is included in the X-ray image 830b, the target may be positioned at a predetermined end part of the object. For example, when a catheter is positioned around the outer boundary 860b of a human body such as a shoulder or a side, the outer boundary 860b of the human body may be included in the X-ray image. Thus, in this case, the X-ray image tracking the region of interest may be usefully applied. Tilting motion, rotational motion, and spherical motion of the C-arm and tilting motion and rotational motion of the table may be usefully applied along with other motions.

Hereinafter, operations of an X-ray apparatus for tracking a region of interest through tilting motion, rotational motion, and spherical motion of a C-arm and tilting motion and rotational motion of a table will be described in more detail with reference to FIGS. 9 through 13.

FIGS. 9A through 9E are diagrams for explaining operations of an X-ray apparatus according to another exemplary embodiment. In more detail, FIG. 9A through FIG. 9E are diagrams for explaining motions of a C-arm including a tilting motion 951 according to an exemplary embodiment.

Referring to FIG. 9A, a target 920 is inserted into a shoulder part of an object 940. FIG. 9B illustrates a scanning environment of the X-ray apparatus that tracks the target 920. FIG. 9D illustrates a scanning environment of the X-ray apparatus that tracks a region of interest. FIG. 9C and FIG. 9E respectively illustrate an X-ray image 970e obtained from detection units 930b and 930d of FIG. 9B and FIG. 9D.

Referring to FIG. 9C, the target 920 is positioned in the center of an X-ray image 970c. For example, a control unit may allow the target 920 to be positioned in the center of the X-ray image 970c by adjusting a position of an X-ray source 960b through longitudinal motion of a C-arm 950b and adjusting a position of the object 940 through lateral motion of a table 900. However, in this case, as shown in FIG. 9C, a large portion of an empty space of the table 900 is included in an X-ray image in an irradiation direction of an X-ray on FIG. 9A. A region of interest 942a including a front side and a rear side of the shoulder part may be efficiently included in the X-ray image. In more detail, a region of interest 942c of the object 940 may not be positioned in the center of the X-ray image 970c.

Therefore, the control unit may move a C-arm 950d to allow tracking of regions of interest 942d and 942e based on a boundary 941 of the object and the table, as shown in FIG. 9D, when capturing an X-ray image 970e. For example, the control unit may tilt the C-arm 950d about 30 degrees in a counterclockwise direction. Referring to FIG. 9E, the region of interest 942e of the object is positioned in the center of the X-ray image 970e after the tilting motion 951 of the C-arm 950d.

A user may more efficiently conduct surgery by using the X-ray image 970e after the tilting motion, rather than the X-ray image 970c before the tilting motion according to a surgical environment.

FIGS. 10A through 10E are diagrams for explaining operations of an X-ray apparatus according to another exemplary embodiment. In more detail, FIG. 10A through FIG. 10E are diagrams for explaining motions of a C-arm including a rotational motion 1051 according to an exemplary embodiment.

Referring to FIG. 10E, a target 1020 is inserted into a shoulder part of an object 1040. FIG. 10B illustrates a scanning environment of the X-ray apparatus that tracks the target 1020. FIG. 10D illustrates a scanning environment of the X-ray apparatus that tracks a region of interest. FIG. 10C and FIG. 10E respectively illustrate an X-ray image 1070e obtained from detection units 1030b and 1030d of FIG. 10 and FIG. 10D.

Referring to FIG. 10C, the target 1020 is positioned in the center of an X-ray image 1070c. For example, a control unit may allow the target 1020 to be positioned in the center of the X-ray image 1070c by adjusting a position of an X-ray source 1060b through longitudinal motion of a C-arm 1050b and adjusting a position of the object 1040 through lateral motion of a table 1000. However, in this case, as shown in FIG. 10C, a large portion of empty space of the table 1000 is included in an X-ray image in an irradiation direction of an X-ray on FIG. 10E. A region of interest 1042a including a side of the shoulder part may be efficiently included in the X-ray image. In more detail, a region of interest 1042c of the object 1040 may not be positioned in the center of the X-ray image 1070c.

Therefore, the control unit may move a C-arm 1050d to allow tracking of regions of interest 1042d and 1042e based on a boundary 1041 of the object and the table, as shown in FIG. 10D, when capturing an X-ray image 1070e. For example, the control unit may rotate the C-arm 1050d about 30 degrees at a reference position. Referring to FIG. 10E, the region of interest 1042e of the object is positioned in the center of the X-ray image 1070e after the rotational motion 1051 of the C-arm 1050d.

A user may more efficiently conduct surgery by using the X-ray image 1070e after the rotational motion 1051, rather than the X-ray image 1070c before the rotational motion according to the surgical environment.

FIGS. 11A through 10E are diagrams for explaining operations of an X-ray apparatus according to another exemplary embodiment. In more detail, FIG. 11A through FIG. 11E are diagrams for explaining motions of a C-arm including spherical motion 1151 according to an exemplary embodiment.

Referring to FIG. 11A, a target 1120 is inserted into a shoulder part of an object 1140. FIG. 11B illustrates a scanning environment of the X-ray apparatus that tracks the target 1120. FIG. 11D illustrates a scanning environment of the X-ray apparatus that tracks a region of interest. FIG. 11C and FIG. 11E respectively illustrate an X-ray image 1170e obtained from detection units 1130b and 1130d of FIG. 11B and FIG. 11D.

Referring to FIG. 11C, the target 1120 is positioned in the center of an X-ray image 1170c. For example, a control unit may allow the target 1120 to be positioned in the center of the X-ray image 1170c by adjusting a position of an X-ray source 1160b through longitudinal motion of a C-arm 1150b and adjusting a position of the object 1140 through lateral motion of a table 1100. However, in this case, as shown in FIG. 11C, a region of interest 1142c of the object 1140 may not be efficiently included in the X-ray image. For example, when both the detection units 1130b and the region of interest 1142c have rectangular shapes, and long sides and short sides do not correspond to each other as shown in FIG. 11C, the region of interest 1142c may not be positioned in the center of the X-ray image 1170c.

Therefore, the control unit may move a C-arm 1150d to allow a region of interest 1142e of the object to be positioned in the center of the X-ray image 1170e as shown in FIG. 11D. For example, the control unit may rotate the C-arm 1150d about 90 degrees at a reference position. That is, the region of interest 1142e of the object corresponds to the detection unit in the long sides and the short sides.

A user may more efficiently conduct surgery by using the X-ray image 1170e after the spherical motion 1151, rather than the X-ray image 1170c before the spherical motion 1151.

FIGS. 12A through 12E are diagrams for explaining operations of an X-ray apparatus according to another exemplary embodiment. In more detail, FIG. 12A through FIG. 12E are diagrams for explaining motions of a table including a tilting motion 1251 according to an exemplary embodiment.

Referring to FIG. 12A, a target 1220 is inserted into a shoulder part of an object 1240. FIG. 12B illustrates a scanning environment of the X-ray apparatus that tracks the target 1220. FIG. 12D illustrates a scanning environment of the X-ray apparatus that tracks a region of interest. FIG. 12C and FIG. 12E respectively illustrate an X-ray image 1270e obtained from detection units 1230b and 1230d of FIG. 12B and FIG. 12D.

Referring to FIG. 12C, the target 1220 is positioned in the center of an X-ray image 1270c. For example, a control unit may allow the target 1220 to be positioned in the center of the X-ray image 1270c by adjusting a position of an X-ray source 1260b through longitudinal motion of a C-arm and adjusting a position of the object 1240 through lateral motion of a table 1250b. However, in this case, as shown in FIG. 12C, a large portion of empty space of the table is included in an X-ray image in an irradiation direction of an X-ray on FIG. 12E. A region of interest 1042a including a front side and a rear side of the shoulder part may be efficiently included in the X-ray image. In more detail, a region of interest 1042c of the object 1040 may not be positioned in the center of the X-ray image 1070c.

Therefore, the control unit may move a table 1250d to allow a region of interest 1242d of the object to be positioned in the center of the X-ray image 1270e as shown in FIG. 12D. For example, the control unit may tilt a table 1250d about 30 degrees in a clockwise direction. Referring to FIG. 12E, the regions of interest 1242d and 1242e of the object are positioned in the center of the X-ray image 1270e after the tilting motion 1251 of the table 1250d.

A user may more efficiently conduct surgery by using the X-ray image 1270e after the tilting motion, rather than the X-ray image 1270c before the tilting motion.

FIGS. 13A through 13E are diagrams for explaining operations of an X-ray apparatus according to another exemplary embodiment. In more detail, FIG. 13A through FIG. 13E are diagrams for explaining motions of a table including rotational motion 1351 according to an exemplary embodiment.

Referring to FIG. 13A, a target 1320 is inserted into a side of an object 1340. FIG. 13B illustrates a scanning environment of the X-ray apparatus that tracks the target 1020. FIG. 13D illustrates a scanning environment of the X-ray apparatus that tracks a region of interest. FIG. 13C and FIG. 13E respectively illustrate an X-ray image 1370e obtained from detection units 1330b and 1330d of FIG. 13B and FIG. 13D.

Referring to FIG. 13C, the target 1020 is positioned in the center of an X-ray image 1370c. For example, a control unit may allow the target 1320 to be positioned in the center of the X-ray image 1370c by adjusting a position of an X-ray source 1360b through longitudinal motion of a C-arm and adjusting a position of the object 1340 through lateral motion of a table 1350b. However, in this case, as shown in FIG. 13C, a large portion of empty space of the table 1350b is included in an X-ray image in an irradiation direction of an X-ray on FIG. 13A. A region of interest 1342a including a side abdomen may be efficiently included in the X-ray image. In more detail, a region of interest 1342c of the object 1340 may not be positioned in the center of the X-ray image 1370c.

Therefore, the control unit may move the table 1350d to allow a region of interest 1342e of the object to be positioned in the center of the X-ray image 1370e as shown in FIG. 13D. For example, the control unit may rotate the table 1350d about 30 degrees in a reference position. Referring to FIG. 13E, the region of interest 1342e of the object is positioned in the center of the X-ray image 1370e after the rotational motion of the table 1350d.

The rotational motion 1351 of the table 1350b in one direction may produce the same effect as that of tilting motion of a C-arm in another direction. That is, the rotational motion 1051 of the C-arm 1550b of FIG. 10A through FIG. 10E may produce the same effect as that of the rotational motion 1351 of the table 1350b of FIG. 13A through FIG. 13E.

A user may more efficiently conduct surgery by using the X-ray image 1370e after the rotational motion, rather than the X-ray image 1370c before the rotational motion.

FIG. 14 is a diagram for explaining an operation of an X-ray apparatus according to an exemplary embodiment. In more detail, FIG. 14 is a diagram for explaining an operation of the user interface unit 650 of FIG. 6 according to an exemplary embodiment.

According to the present exemplary embodiment, the user interface unit 650 may receive a first input 1450. In this regard, the first input 1450 may be a user input for selecting at least one of the C-arm 630 and the table 640, and setting a sequence of movements with respect to the selected object. The control unit 620 may move at least one of the C-arm 630 and the table 640 to allow a target to be included in an X-ray image based on the first input 1450 and position information of the target.

The user interface unit 650 may receive the first input 1450 through a user interface screen 1400 displayed by the display unit 680. For example, the user interface screen 1400 may include a first icon 1410 moving a C-arm only, a second icon 1420 moving a table only, a third icon 1430 moving the table after moving the C-arm, and a fourth icon 1440 moving the C-arm after moving the table.

A user may select the most efficient control method according to the surgical environment through the first input 1450 selecting one of the above icons. The control unit 620 may automatically move the C-arm 630 and/or the table 640 based on the first input 1450 and the position information of the target. Thus, the user does not need to personally move the C-arm and/or the table to track the target.

When the user selects the third icon 1430 and the fourth icon 1440, the user may set a movement distance ratio for setting how much to move the C-arm and the table. In more detail, the user may set a ratio of a longitudinal motion distance of the C-arm with respect to a longitudinal motion distance of the table through Rx 1460, and may set a ratio of a lateral motion distance of the C-arm with respect to a longitudinal motion distance of the table through Ry 1470.

Referring to FIG. 14, the user selects the third icon 1430 through the first input 1450 and sets Rx and Ry as 0.5. Thus, the control unit 620 may move the C-arm by the same movement distance after moving the table first so as to track the target.

The user interface unit 650 of the present exemplary embodiment may receive a third input 1490 for stopping a movement of the C-arm 630 or the table 640. The control unit 620 may stop the movement of the C-arm 630 or the table 640 based on the third input 1490.

In more detail, the user interface screen 1400 may further include a fifth icon 1480 for stopping an operation of the C-arm or the table that is currently moving. The user may stop the movement of the C-arm 630 or the table 640 through the fifth icon 1480.

FIG. 15 is a diagram for explaining an operation of an X-ray apparatus according to another exemplary embodiment. In more detail, FIG. 15 is a diagram for explaining an operation of the user interface unit 650 of FIG. 6 according to another exemplary embodiment.

According to the present exemplary embodiment, the user interface unit 650 may receive second inputs 1530, 1531, and 1532 for selecting at least one of a C-arm and a table to be controlled, and setting a control sequence of the selected object. A control unit may control at least one of the C-arm and the table to allow tracking of a target based on the second inputs 1530, 1531, and 1532 and position information of the target, when capturing an X-ray image.

The user interface unit 650 may receive the second inputs 1530, 1531, and 1532 through a user interface screen 1500 displayed by the display unit 680. For example, the user interface screen 1500 may include a lateral motion icon 1511, a longitudinal motion icon 1512, a tilting motion icon 1513, a rotational motion icon 1514, and a spherical motion icon 1515 of the C-arm. The user interface screen 1500 may include a lateral motion icon 1521, a longitudinal motion icon 1522, a tilting motion icon 1523, and a rotational motion icon 1524 of the table.

A user may select the most efficient control method according to the surgical environment through the second inputs 1530, 1531, and 1532 selecting one of the above icons. The control unit 620 may automatically move the C-arm 630 and/or the table 640 based on the second inputs 1530, 1531, and 1532 and the position information of the target. Thus, the user himself/herself does not need to move the C-arm and/or the table to track the target.

The user interface unit 650 may determine the control sequence according to a sequence of the second inputs 1530, 1531, and 1532. For example, in FIG. 15, when the user sequentially enters the second input 1530 selecting the lateral motion icon 1511 of the C-arm, the second input 1531 selecting the tilting motion icon 1513 of the C-arm, and the second input 1532 selecting the longitudinal motion icon 1522 of the table, the control unit may move the C-arm laterally first, then tilt the C-arm, and finally move the table longitudinally.

The user interface unit 650 of the present exemplary embodiment may receive a third input for stopping a movement of the C-arm 630 or the table 640. The control unit 620 may stop the movement of the C-arm 630 or the table 640 based on the third input.

In more detail, the user interface screen 1500 may further include a C-arm stop icon 1516 and a table stop icon 1526 for stopping operations of the C-arm or the table that are currently moving. The user may stop the movements of the C-arm 630 or the table 640 through the C-arm stop icon 1516 and the table stop icon 1526.

Hereinafter, an operation in which the data obtaining units 510 and 610 obtain position information of a target based on electrode signals detected from a plurality of electrodes according to an exemplary embodiment will be described in detail with reference to FIGS. 16 and 17.

FIG. 16 is a diagram for explaining operations of an X-ray apparatus according to an exemplary embodiment.

The data obtaining units 510 and 610 according to an exemplary embodiment may include a plurality of electrocardiogram (ECG) measurement electrodes 1630, 1640, 1650, 1660, 1670, and 1680 attached to an object and may obtain position information of the object in a target based on ECG signals 1631, 1641, 1636, and 1646 detected from the ECG measurement electrodes 1630, 1640, 1650, 1660, 1670, and 1680.

The X-ray apparatuses 500 and 600 according to an exemplary embodiment may track the target according to a user's intention to generate an X-ray or a fluoroscopy image by using the ECG measurement electrodes 1630, 1640, 1650, 1660, 1670, and 1680 without any additional element besides elements of a general X-ray apparatus or special manipulation by the user. Accordingly, the X-ray apparatuses 500 and 600 may provide a more efficient environment to the user. The X-ray apparatuses 500 and 600 may measure ECG of the object or a patient and monitor medical surgery such as angiography and surgical treatment by using the ECG measurement electrodes 1630, 1640, 1650, 1660, 1670, and 1680. Thus, the X-ray apparatuses 500 and 600 may provide a more safe and efficient environment to the user and the object.

Referring to FIG. 16, the two electrodes 1630 and 1640 may be attached to positions corresponding to an X axis, the two electrodes 1650 and 1660 may be attached to positions corresponding to a y axis, and the two electrodes 1670 and 1680 may be attached to positions corresponding to a z axis, among the ECG measurement electrodes 1630, 1640, 1650, 1660, 1670, and 1680. A first coordinate system 1620 may be a 3-dimensional (3D) rectangular coordinate system based on the ECG measurement electrodes 1630, 1640, 1650, 1660, 1670, and 1680.

FIG. 16A illustrates the ECG signals 1631 and 1641 detected from the ECG measurement electrodes 1630, 1640, 1650, 1660, 1670, and 1680 before the target is inserted into the object.

For example, the ECG signals 1631 and 1641 detected from the ECG measurement electrodes 1630, 1640, 1650, 1660, 1670, and 1680 of FIG. 16A may be stable. Thus, the data obtaining units 510 and 610 may determine that no target is present in the object.

FIG. 16B illustrates the ECG signals 1636 and 1646 detected from the ECG measurement electrodes 1630, 1640, 1650, 1660, 1670, and 1680 after the target is inserted into the object.

For example, there may be a change in the ECG signals 1636 and 1646 detected from the ECG measurement electrodes 1630, 1640, 1650, 1660, 1670, and 1680 of FIG. 16B. In more detail, there may be a relatively large change in the ECG signal 1636 detected from the ECG measurement electrode 1630 positioned closer to the target, whereas there may be a relatively small change or no change in the ECG signal 1646 detected from the ECG measurement electrode 1640 positioned farther from the target.

Therefore, the data obtaining units 510 and 610 may obtain the position information of the target based on the ECG signals 1631, 1641, 1636, and 1646 detected from the ECG measurement electrodes 1630, 1640, 1650, 1660, 1670, and 1680. In more detail, the position information of the target may be obtained based on a change in the ECG signals 1631, 1641, 1636, and 1646 that may occur due to insertion of the target or a movement.

According to the present exemplary embodiment, the data obtaining units may measure impedance of the object included in a region of interest based on an electrode signal and obtain the position information based on the impedance of the object.

For example, the data obtaining units 510 and 610 may measure the impedance of the object through current and voltage detected from the ECG measurement electrodes 1630, 1640, 1650, 1660, 1670, and 1680, and may obtain the position information of the target based on a change in the impedance of the object with respect to the target.

The ECG measurement electrodes 1630, 1640, 1650, 1660, 1670, and 1680 may be attached to the object in such a manner that a region of interest 1610 may be included in the ECG measurement electrodes 1630, 1640, 1650, 1660, 1670, and 1680 to obtain the exact position information of the target.

FIGS. 17A and 17B are diagrams for explaining operations of an X-ray apparatus according to another exemplary embodiment. In more detail, FIG. 17A and FIG. 17B illustrate operations in which the data obtaining units 510 and 610 of the X-ray apparatuses 300 and 400 obtain position information of a target by using impedance maps 1710 and 1720.

According to an exemplary embodiment, the data obtaining units 510 and 610 may measure impedance of an object included in a region of interest based on an electrode signal, generate the impedance maps 1710 and 1720 of the object based on the impedance of the object, and obtain the position information of the target based on the impedance maps 1710 and 1720 of the object.

The data obtaining units 510 and 610 of FIG. 16A through FIG. 17B may correspond to each other in an operation of measuring the impedance of the object. Thus, redundant descriptions therebetween are omitted.

The impedance maps 1710 and 1720 may be data obtained by subdividing the object and measuring a size of the impedance with respect to a part of each of the subdivided objects.

For example, the impedance maps 1710 and 1720 may express the size of the impedance with respect to the part of each of the subdivided objects. In more detail, the larger the size of the impedance with respect to a part of a predetermined object, the darker the color of the impedance map corresponding to the part of the object may be expressed.

FIG. 17A illustrates the impedance map 1710 of the object before the target is inserted into the object. For example, impedances of parts 1711 and 1717 of the object included in a region of interest of FIG. 17A may be constant. Thus, the data obtaining units 510 and 610 may express the impedance map 1710 of FIG. 17A in one color.

FIG. 17B illustrates the impedance map 1720 of the object after a target 1730 is inserted into the object. For example, a size of impedance of a part 1721 of the object closer to the object may be relatively large, whereas a size of impedance of a part 1722 of the object farther from the object may be relatively small. Thus, the data obtaining units 510 and 610 may express the impedance map 1720 of FIG. 17B in a plurality of colors.

However, an operation in which the data obtaining units 510 and 610 generate the impedance maps 1710 and 1720 is not limited to that described above. The impedance maps may include all types of data obtained by measuring a size of impedance of each part of an object such as a color, a volume real number, a complex number, etc.

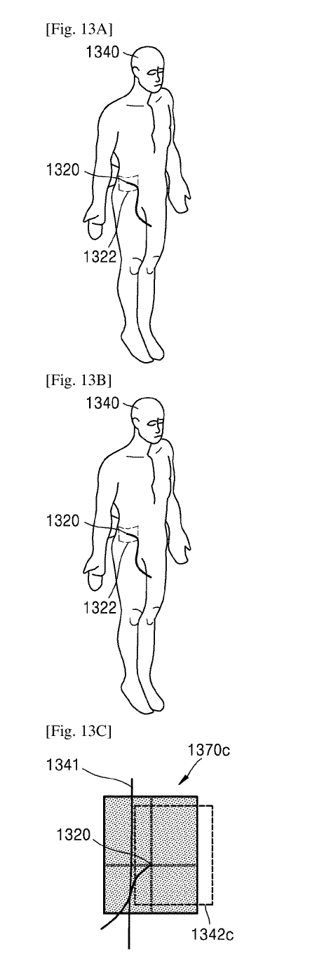

Hereinafter, a method in which the control units 520 and 620 according to exemplary embodiments set a position of a target with respect to an X-ray image based on position information of the target will be described in detail with reference to FIGS. 18 through 25.

FIG. 18 is a diagram for explaining operations of the X-ray apparatuses 300 and 400 according to an exemplary embodiment. In more detail, FIG. 18 illustrates the operations of the X-ray apparatuses 300 and 400 before a target is inserted into an object 1800.

According to an exemplary embodiment, a control unit may set first coordinates indicating a position of the target on a first coordinate system with respect to the object based on position information of the target. The control unit may transform the first coordinates into second coordinates on a second coordinate system with respect to an X-ray image and move at least one of a C-arm and a table to allow tracking of the target based on the second coordinates when capturing the X-ray image.

In more detail, a first coordinate system 1840 of FIG. 18 may be a coordinate system with respect to the object 1800. That is, the first coordinate system 1840 may be a reference for setting first coordinates (not shown) indicating an absolute position of a target (not shown) in the object.

According to an exemplary embodiment, an origin and an axis of the first coordinate system 1840 may be determined based on a plurality of electrodes (not shown) attached to the object 1800. For example, the first coordinate system of FIG. 18 may be a 3D rectangular coordinate system based on the plurality of electrodes attached to positions corresponding to three axes 1841, 1842, and 1843 that are perpendicular to each other.

The plurality of electrodes may include regions of interest and may be attached to the object in such a manner that the first coordinate system 1840 may include all the regions of interest.

A second coordinate system 1860 of FIG. 18 may be a coordinate system with respect to the X-ray image. That is, the second coordinate system 1860 may be a reference for setting second coordinates (not shown) indicating a position of a target included in the X-ray image.

According to an exemplary embodiment, the second coordinate system 1860 may be based on an X-ray scanning environment. For example, the second coordinate system 1860 of FIG. 18 may be a 2D rectangular coordinate system with respect to a 2D X-ray scanning image. An origin of the second coordinate system 1860 of FIG. 18 may be positioned on a predetermined plane 1870 corresponding to the X-ray image.

Therefore, the X-ray apparatuses 300 and 400 may more efficiently adjust a C-arm and/or a table based on the second coordinates that indicate a relative position of the target rather than the first coordinates that indicate an absolute position of the target to allow tracking of the target when capturing the X-ray image. The second coordinate system that is a reference of the second coordinates is based on the X-ray image, and thus the control unit may more efficiently calculate f(x, y) of Equation 1 described above based on the second coordinate system rather than the first coordinate system.

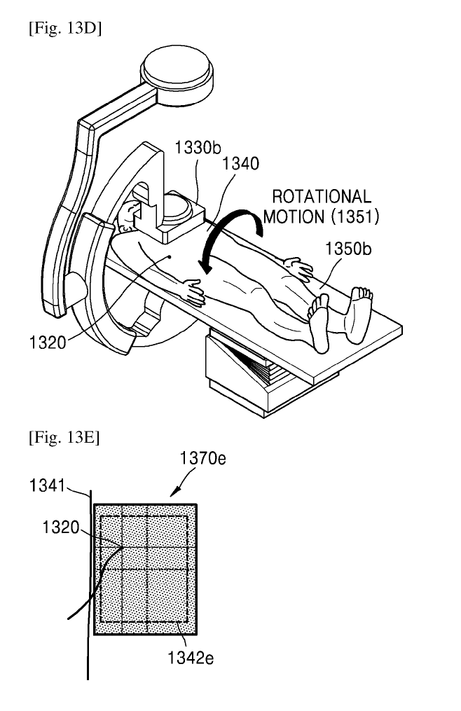

FIG. 19 is a diagram for explaining operations of the X-ray apparatuses 500 and 600 according to another exemplary embodiment. In more detail, FIG. 19 illustrates the operations of the X-ray apparatuses 500 and 600 after a target 1910 is inserted into an object. A first coordinate system 1970 and a second coordinate system 1980 of FIG. 19 may correspond to the first coordinate system 1840 and the second coordinate system 1860 of FIG. 18. Thus, redundant descriptions between FIGS. 19 and 5 are omitted.

According to an exemplary embodiment, the control units 520 and 620 of the X-ray apparatuses 500 and 600 may set a first coordinates 1900 indicating a position of the target 1910 on the first coordinate system 1970 with respect to the object based on position information of the target 1910. For example, the control units 520 and 620 may set the first coordinates 1900 of FIG. 19 as (x1, y1, z1).

The first coordinates 1900 may more accurately indicate a position of a target based on an irradiation region of an X-ray by a collimator. In more detail, the narrower the irradiation region of the X-ray, the more minutely the first coordinate system 1970 may be divided to accurately track the target, and thus the first coordinates 1900 may more accurately indicate the position of the target. That is, according to an exemplary embodiment, a user may adjust a size of the irradiation region of the X-ray, and the control units 520 and 620 may adjust a degree of minuteness of the first coordinate system 1970 based on the irradiation region of the X-ray.

According to an exemplary embodiment, the control units 520 and 620 of the X-ray apparatuses 500 and 600 may transform the first coordinates 1900 into second coordinates 1950 on a second coordinate system 1980 with respect to an X-ray image. For example, the control units 520 and 620 may set the second coordinates 1950 as (a1, b1) by transforming the first coordinates 1900 (x1, y1, z1) of FIG. 19. As described above, the second coordinate 1950 may indicate a position 1960 of the target appearing on the X-ray image.

In more detail, the control units 520 and 620 may transform the first coordinates 1900 into the second coordinates 1950 through a geometric coordinate transformation matrix (hereinafter referred to as “M”) as shown in Equation 5 below.

$$[ \begin{matrix}a 1 \\ b 1 \\ c 1 \\ 1\end{matrix} ] = M [ \begin{matrix}x 1 \\ y 1 \\ z 1 \\ 1\end{matrix} ] \qquad [ Equation 5 ]$$

According to an exemplary embodiment, the control units 520 and 620 may set c1 in order to match dimensions of the first coordinates 1900 and the second coordinates 1950. Thus, c1 may be an arbitrary value for convenience of calculation. For example, c1 may be 0 and may have the same value as z1.

That is, the control units 520 and 620 may not specify a value of c1 by transforming the 3D first coordinates 1900 of FIG. 19 into the 2D second coordinates 1950.

According to an exemplary embodiment, M may be defined as shown in Equation 6 below.

$$M = T \cdot S \cdot {R}_{x} \cdot {R}_{y} \cdot {R}_{z} \\ T \cdot S = [ \begin{matrix}{S}_{x} & 0 & 0 & Δ x \\ 0 & {S}_{y} & 0 & Δ y \\ 0 & 0 & {S}_{z} & Δ z \\ 0 & 0 & 0 & 1\end{matrix} ] \\ {R}_{x} = [ \begin{matrix}1 & 0 & 0 & 0 \\ 0 & cos α & sin α & 0 \\ 0 & - sin α & cos α & 0 \\ 0 & 0 & 0 & 1\end{matrix} ] \\ {R}_{y} = [ \begin{matrix}cos β & 0 & - sin β & 0 \\ 0 & 1 & 0 & 0 \\ sin β & 0 & cos β & 0 \\ 0 & 0 & 0 & 1\end{matrix} ] \\ {R}_{z} = [ \begin{matrix}cos γ & sin γ & 0 & 0 \\ - sin γ & cos γ & 0 & 0 \\ 0 & 0 & 1 & 0 \\ 0 & 0 & 0 & 1\end{matrix} ] \qquad [ Equation 6 ]$$

According to an exemplary embodiment, a transition matrix T may be a matrix for transiting an origin of the first coordinate system 1970 into an origin of the second coordinate system 1980. In more detail, T may move the first coordinates 1900 by (Δx, Δy, Δz).

A scale matrix S may be a matrix for transiting a scale of x, y, and z axes of the first coordinate system 1970 into a scale of x, y, and z axes of the second coordinate system 1980. In more detail, S may respectively transit the x, y, and z axes by Sx, Sy, and Sz.

Rotation matrixes Rx, Ry, and Rz may be matrixes for respectively rotating the first coordinate system 1970 in a clockwise direction with respect to the x, y, and z axes. In more detail, the first coordinate system 1970 may respectively rotate in the clockwise direction by α, β, and γ with respect to the x, y, and z axes through Equation 6.

For example, when the first coordinates 1900 and the second coordinates 1950 are identical to each other, M may be a unit matrix.

As another example, when the first coordinate system 1970 and the second coordinate system 1980 are identical to each other in x and y axial directions, and are different from each other in terms of the position of an origin and the scale of an axis, the control units 520 and 620 may set the second coordinates 1950 as shown in Equation 7 below.

$$[ \begin{matrix}a 1 \\ b 1 \\ c 1 \\ 1\end{matrix} ] = T \cdot S \cdot I \cdot [ \begin{matrix}x 1 \\ y 1 \\ z 1 \\ 1\end{matrix} ] \\ a 1 = {S}_{x} \cdot x 1 + Δ x \\ b 1 = {S}_{y} \cdot y 1 + Δ y \qquad [ Equation 7 ]$$

As another example, when the first coordinate system 1970 and the second coordinate system 1980 are different from each other in the x and y axial directions, and are different from each other in terms of the position of the origin and the scale of the axis, the control units 520 and 620 may set the second coordinates 1950 as shown in Equation 8 below.

$$[ \begin{matrix}a 1 \\ b 1 \\ c 1 \\ 1\end{matrix} ] = T \cdot S \cdot {R}_{z} \cdot [ \begin{matrix}x 1 \\ y 1 \\ z 1 \\ 1\end{matrix} ] \\ a 1 = {S}_{x} \cdot ( cos γ \cdot x 1 + sin γ \cdot y 1 ) + Δ x \\ b 1 = {S}_{y} \cdot ( - sin γ \cdot x 1 + sin γ \cdot y 1 ) + Δ y \qquad [ Equation 8 ]$$

As another example, the control units 520 and 620 may rotate the first coordinate system 1970 of FIG. 19 in a counterclockwise direction by 90° (γ=−90°) with respect to the z axis, transit the origin, transfer the scale, and set the second coordinate 1950. That is, the control units 520 and 620 may set the second coordinates 1950 as shown in Equation 9 below.

a1=Sₓ·y1+Δx

b1=S_{y}·x1+Δy [Equation 9]

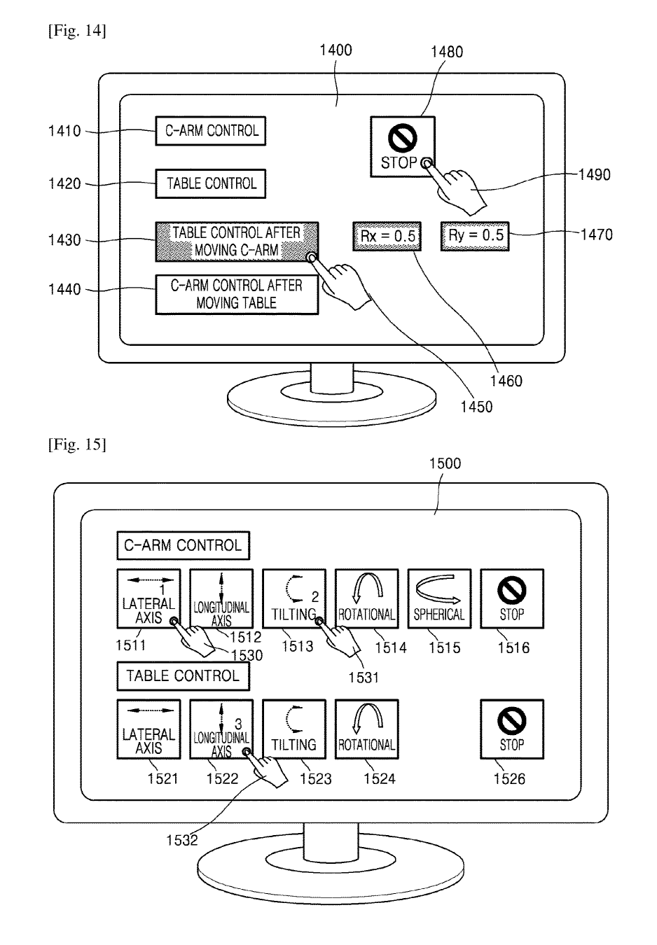

However, the method in which the control units 520 and 620 set the second coordinates 1950 is not limited thereto. Other methods in which the control units 520 and 620 set the second coordinates 1950 will be described in detail with reference to FIGS. 20 and 21.

The control units 520 and 620 may set M based on Equation 10 below according to an exemplary embodiment.

Arg_{M}min(DATA2−M·DATA1) [Equation 10]

However, DATA1 may be data of the first coordinate system, and DATA2 may be data of the second coordinate system. That is, the control units 520 and 620 may set M as a value for minimizing a feature difference between the data of the second coordinate system and the data transformed from the first coordinate system.

For example, DATA1 may be an origin of the first coordinate system, and DATA2 may be an origin of the second coordinate system. That is, the control units 520 and 620 may set M as a value for minimizing a feature difference between the origin DATA2 of the second coordinate system and M?DATA1 transformed from the origin DATA1 of the first coordinate system.

As another example, DATA1 and DATA2 may be values previously set by a user based on a scanning environment.

In more detail, according to an exemplary embodiment, the control units 520 and 620 may use a 1 norm based pseudo inverse solution, a 2 norm based gradient method, etc. so as to calculate M.

FIG. 20 is a diagram for explaining operations of the X-ray apparatuses 500 and 600 according to another exemplary embodiment. In more detail, like FIG. 19, FIG. 20 illustrates the operations of the X-ray apparatuses 500 and 600 after a target 2010 is inserted into an object. However, FIG. 20 illustrates other operations in which the control units 520 and 620 set first and second coordinates according to an exemplary embodiment.

According to an exemplary embodiment, the control units 520 and 620 may set a first coordinate system 2011 as a 3D rectangular coordinate system based on electrode signals detected from a plurality of electrodes attached to positions corresponding to three axes that are perpendicular to each other. The control units 520 and 620 may set second coordinate systems 2012 and 2013 as 2D rectangular coordinate systems that are planes 2020 and 2050 perpendicular to an irradiation direction 2011 of an X-ray. The control units 520 and 620 may set points 2040 and 2070 on the planes 2020 and 2050 closest to the first coordinates 2000 as the second coordinates 2030 and 2060.

According to an exemplary embodiment, the control units 520 and 620 may set a value of the first coordinates 2000 of FIG. 20 as (x1, y1, z1).

According to an exemplary embodiment, the first coordinate system 2011 of FIG. 20 that is a reference of the first coordinates 2000 may be independent from the irradiation direction 2011 of the X-ray, whereas the second coordinate systems 2012 and 2013 of FIG. 20 that are references of the second coordinates 2030 and 2060 may be dependent upon the irradiation direction 2011 of the X-ray. Thus, the control units 520 and 620 may more efficiently track the target based on the second coordinates and move the C-arms 530 and 630 and/or the tables 540 and 640.

In more detail, origins of the second coordinate systems 2012 and 2013 of FIG. 20 may be positioned on the planes 2020 and 2050 perpendicular to the irradiation direction 2011 of the X-ray. The origins of the second coordinate systems 2012 and 2013 may be positioned at any points of the corresponding planes 2020 and 2050 based on an X-ray source and a scanning environment of the X-ray including a position of the object.

Detailed positions of the planes 2020 and 2050 may be set based on the scanning environment of the X-ray. For example, the planes 2020 and 2050 may be positioned in a detection unit (not shown) of the X-ray apparatus. FIG. 20 illustrates examples of the planes 2020 and 2050 and the second coordinate systems 2012 and 2013.

According to an exemplary embodiment, the second coordinate systems 2012 and 2013 of FIG. 20 may include two axes perpendicular to each other on the corresponding planes 2020 and 2050. Thus, the second coordinate systems 2012 and 2013 of FIG. 20 may be 2D rectangular coordinate systems on the corresponding planes 2020 and 2050.

According to an exemplary embodiment, the control units 520 and 620 may set the second coordinates 2030 and 2060 as points 2040 and 2070 on the planes 2020 and 2050 closest to the first coordinates 2000. In more detail, the control units 520 and 620 may set the second coordinates 2030 and 2060 as the points 2040 and 2070 in which a line 2002 passing by the first coordinates 2000 and extending in the same direction as the irradiation direction 2001 of the X-ray meets the planes 2020 and 2050.

In this regard, since the line 2002 is the same as the irradiation direction 2001 of the X-ray, the line 2002 may be perpendicular to the planes 2020 and 2050. Thus, the second coordinates 2030 and 2060 of FIG. 20 are the points 2040 and 2070 on the planes 2020 and 2050 closest to the first coordinates 2000.

According to an exemplary embodiment, the control units 520 and 620 may set values of the second coordinates 2030 and 2060 of FIG. 20 as (a1, b1) or (a2, b2).

The second coordinates 2030 and 2060 of FIG. 20 may be the same points as the second coordinates 1950 of FIG. 19.

FIG. 21 is a diagram for explaining operations of the X-ray apparatuses 500 and 600 according to another exemplary embodiment. In more detail, like FIG. 20, FIG. 21 illustrates the operations of the X-ray apparatuses 500 and 600 after a target 2110 is inserted into an object. However, FIG. 21 illustrates other operations in which the control units 520 and 620 set first and second coordinates according to an exemplary embodiment.

According to an exemplary embodiment, the control units 520 and 620 may set a first coordinate system 2101 as a 3D rectangular coordinate system and a second coordinate system 2102 as a 3D rectangular coordinate system including an axis 2103 in the same direction as an irradiation direction 2121 of an X-ray, based on electrode signals detected from a plurality of electrodes attached to positions corresponding to three axes that are perpendicular to each other.

In more detail, the control units 520 and 620 may set the axis 2103 in the same direction as the irradiation direction 2121 of the X-ray as a z axis of the second coordinate system 2102 of FIG. 21. Thus, second coordinates 2120 of FIG. 21 may indicate an absolute position of a target in an object like first coordinates 2100. That is, the second coordinates 2120 of FIG. 21 may be an actual position of the target in the object.

For example, the control units 520 and 620 may set a value of the second coordinates 2120 of FIG. 21 as (a1, b1, c1). In this regard, the control units 520 and 620 may set c1 that is an element of a z axis of the second coordinates 2120 of FIG. 21 as a value corresponding to the actual position of the target.

However, according to an exemplary embodiment, the second coordinates 2120 of FIG. 21 may be still dependent upon the irradiation direction 2121 of the X-ray, and thus the control units 520 and 620 may more efficiently move the C-arms 530 and 630 and/or the tables 540 and 640 based on the second coordinates rather than the first coordinates.

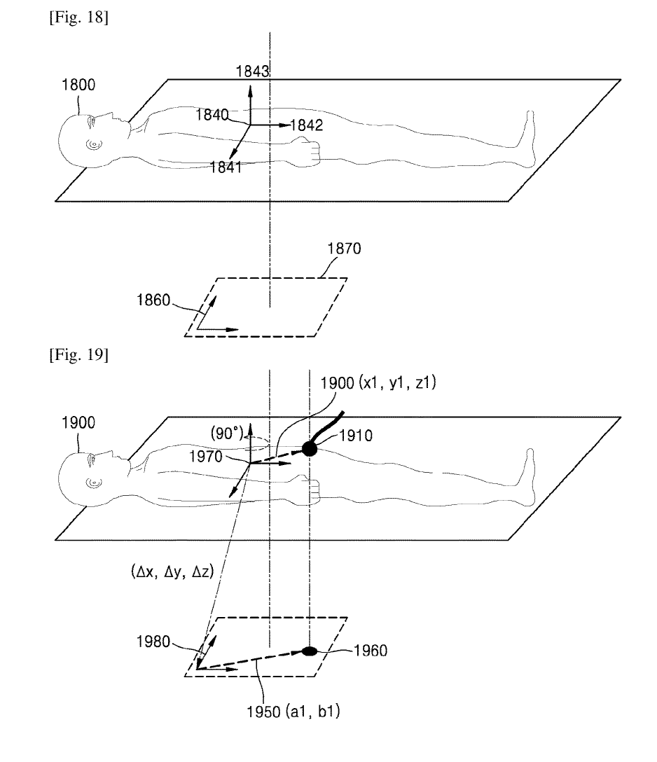

FIG. 22 is a diagram for explaining operations of the X-ray apparatuses 500 and 600 according to another exemplary embodiment. In more detail, FIG. 22 illustrates the operations of the X-ray apparatuses 500 and 600 after the target 2110 is inserted into an object, like FIG. 20. However, FIG. 22 illustrates other operations in which the control units 520 and 620 set first coordinates 2240 and second coordinates 2280 according to an exemplary embodiment.

According to an exemplary embodiment, the data obtaining units 510 and 610 may obtain position information of an object 2220 based on an impedance map 2320. The control units 520 and 620 may set the first coordinates 2240 and the second coordinates 2280 based on the position information.

The operations in which the data obtaining units 510 and 510 generate the impedance map of the object in FIG. 22 may correspond to the operations in FIG. 17. The operations in which the control units 520 and 620 set the first coordinates 2240 and the second coordinates 2280 in FIG. 22 may correspond to the operations in FIGS. 18 through 21. Thus, redundant descriptions between FIGS. 22 and 17 through 21 are omitted.

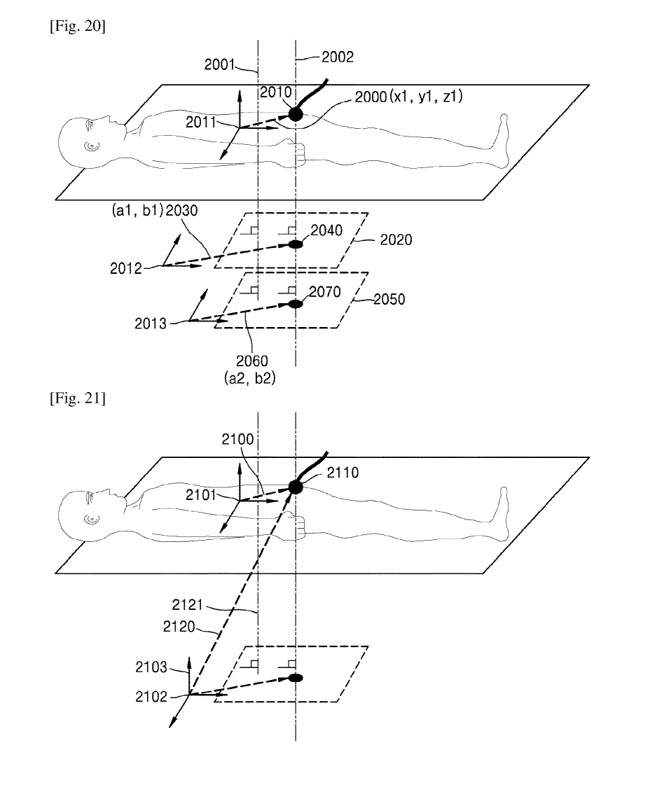

FIG. 23 is a diagram for explaining operations of X-ray apparatuses according to another exemplary embodiment. In more detail, FIG. 23 illustrates operations in which the control units 520 and 620 of the X-ray apparatuses including a C-arm 2300 tilting at 45 degrees set M of Equation 5. A first coordinate system 2330 of FIG. 23 may correspond to the first coordinate system 1840 of FIG. 18. Thus, redundant descriptions between FIGS. 23 and 18 are omitted.

According to an exemplary embodiment, the control units 520 and 620 may specify an irradiation direction 2320 of an X-ray based on an angle 2310 of the C-arm 2300. The control units 520 and 620 may more efficiently set M based on the irradiation direction 2320 of the X-ray. In this regard, the angle 2310 of the C-arm 2300 may be an angle formed by a reference line 2321 and the irradiation direction 2320 of the X-ray according to a movement of the C-arm 2300.

In more detail, the control units 520 and 620 may set a second coordinate system as a 2D rectangular coordinate system that is a plane 2360 perpendicular to the irradiation direction 2320 of the X-ray. The control units 520 and 620 may set a point in which a line 2330 passing through DATA1 2340 of Equation 10 and parallel to the irradiation direction 2320 of the X-ray meets the plane 2360 as DATA2 2350.

As described above, DATA1 may be data of the first coordinate system. For example, DATA1 may be an origin of the first coordinate system. As another example, DATA1 may be a predetermined point on the first coordinate system set by a user.

The control units 520 and 620 may set DATA2 on the plane 2360 that is the second coordinate system 2370, and thus the DATA2 2350 may be data of the second coordinate system 2370.

The control units 520 and 620 may set M as a value that minimizes a feature difference between the DATA2 and data transformed from the DATA1 based on Equation 10. In more detail, according to an exemplary embodiment, the control units 520 and 620 may use a 1 norm based pseudo inverse solution method or a 2 norm based gradient method in order to obtain M.

As described above, a position of the target 2340 may be more accurately obtained by adjusting a reference plane 2360 of at least one of the first coordinate system 2380 and the second coordinate system 2370 based on the angle of the C-arm 2300. In more detail, the position of the target 2340 may be more accurately obtained by adjusting the reference plane 2360 of the second coordinate system 2370 as a plane perpendicular to the irradiation direction 2320 of the X-ray based on the angle of the C-arm 2300.

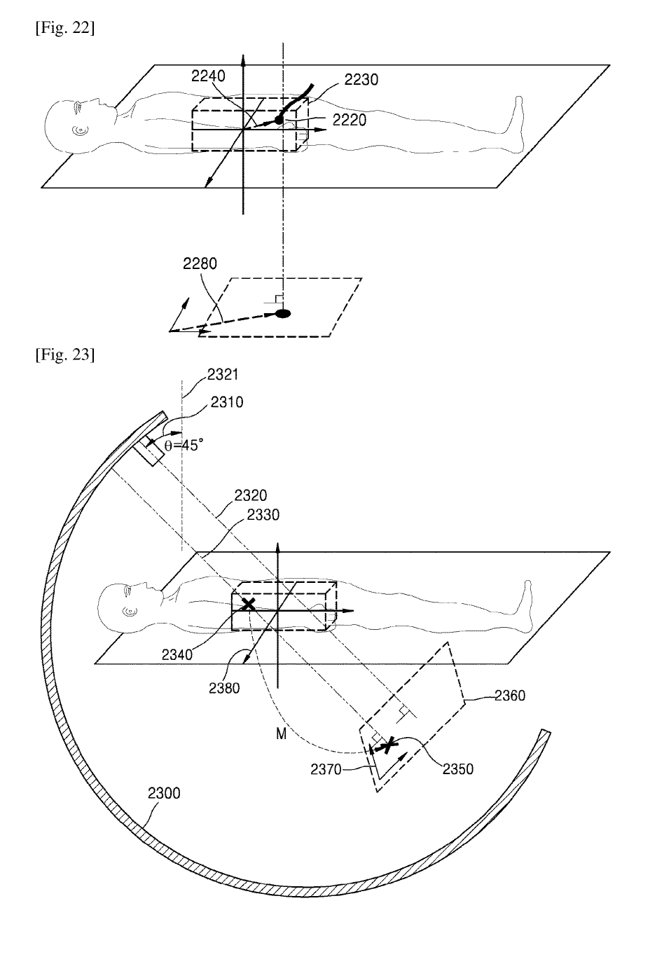

FIG. 24 is a diagram for explaining operations of X-ray apparatuses according to another exemplary embodiment. In more detail, FIG. 24 illustrates other operations in which the control units 520 and 620 of the X-ray apparatuses including a C-arm 2400 tilting at 45 degrees set M. A first coordinate system 2430 of FIG. 24 may correspond to the first coordinate system 1840 of FIG. 18. Thus, redundant descriptions between FIGS. 24 and 18 are omitted.

The first coordinate system 2430 and a second coordinate system 2470 of FIG. 24 may correspond to the first coordinate system 2380 and the second coordinate system 2370 of FIG. 23. Thus, redundant descriptions between FIGS. 24 and 23 are omitted.

According to an exemplary embodiment, DATA1 and DATA2 of Equation 10 may be set in advance. For example, a user may personally set DATA1 and DATA 2, and the control units 520 and 620 may set M that minimizes a feature difference between data transformed from DATA1 and DATA2. As described above, the control units 520 and 620 may specify an irradiation direction 2420 of an X-ray based on an angle 2411 of the C-arm 2400 and may more efficiently set M based on the irradiation direction 2420 of the X-ray.

In more detail, the user may set DATA1 2440 that is data on the first coordinate system 2430 and DATA2 2455 that is data on the second coordinate system 2470. According to an exemplary embodiment, the control units 520 and 620 may set a point in which a line 2431 passing through the DATA1 2440 and parallel to the irradiation direction 2420 of the X-ray meets the plane 2460 as DATA 3 2450. In this regard, the control units 520 and 620 may set the DATA3 2450 on the second coordinate system 2470 that is the plane 2460, and thus the DATA3 2450 may be data of the second coordinate system 2470.

According to an exemplary embodiment, the control units 520 and 620 may set more efficiently M in consideration of a feature difference between the DATA2 2455 and the DATA3 2450.

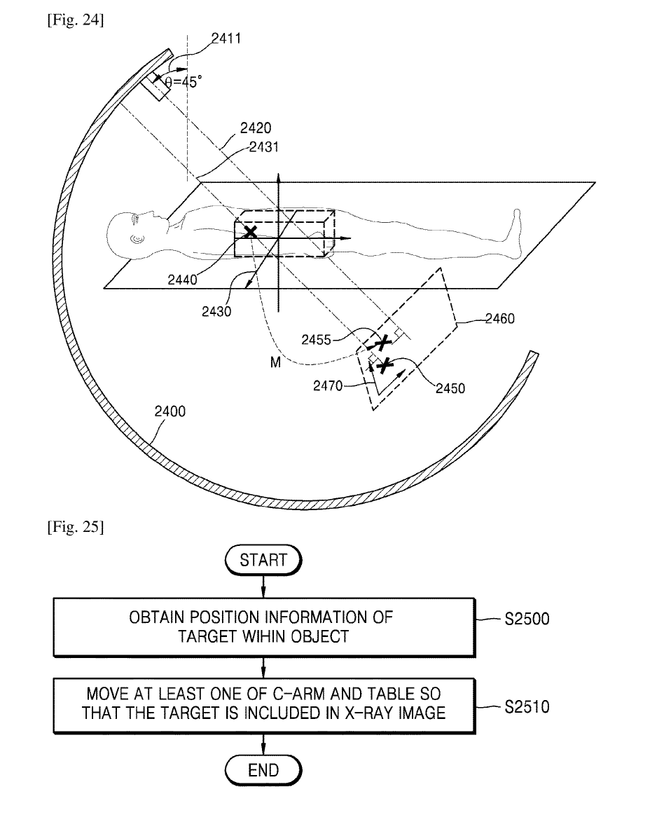

FIG. 25 is a flowchart of an X-ray scanning method according to an exemplary embodiment. In more detail, FIG. 25 shows the X-ray scanning method of generating a fluoroscopy image by automatically moving at least one of a C-arm and a table and tracking a target. The X-ray scanning method of the present exemplary embodiment may be performed by using the X-ray apparatuses 500 and 600 described with reference to FIGS. 5 and 6 above. Operations of the X-ray scanning method include the same technical idea as that of the operations of the X-ray apparatuses 500 and 600 described with reference to FIGS. 5 and 6 above. Thus, redundant descriptions between FIGS. 25 and 5 through 24 are omitted.

Referring to FIG. 25, the X-ray scanning method of the present exemplary embodiment may obtain position information of a target included in an object (operation S2500). Operation S2500 may be performed by the data obtaining units 510 and 610 of the X-ray apparatuses 500 and 600.

Operation S2500 may obtain the position information of the target based on electrode signals detected from a plurality of electrodes attached to the object.

The X-ray scanning method of the present exemplary embodiment may move at least one of a C-arm that adjusts a position of an X-ray source and a table on which the object is positioned to allow tracking of the target when capturing the X-ray image (operation S2510). Operation S2510 may be performed by the control units 520 and 620 of the X-ray apparatuses 500 and 600.

Operation S2510 may move at least one of the C-arm and the table to allow the target to be positioned in the center of the X-ray image based on the position information of the target.

Operation S2510 may move at least one of the C-arm and the table to allow tracking of at least one of the target and a region of interest when capturing the X-ray image.

Operation S2510 may enable the target to be positioned within a predetermined distance from the center of the X-ray image based on the position information of the target, recognize a boundary between the object and the table, and move at least one of the C-arm and the table to track the region of interest based on the boundary.

The C-arm adjusts a position of the X-ray source through at least one of longitudinal motion, lateral motion, tilting motion, rotational motion, and spherical motion and operation S2510 may control at least one of the motions of the C-arm to track the target based on the position information of the target.

The table adjusts a position of the object through at least one of longitudinal motion, lateral motion, tilting motion, and rotational motion and operation S2510 may control at least one of the motions of the table to track the target based on the position information of the target.

As described above, according to the one or more of the above exemplary embodiments, an X-ray apparatus may generate an X-ray image or a fluoroscopy image that automatically moves at least one of a C-arm and a table to track a target based on position information of the target. Thus, a dose of radiation exposed to the object may be minimized. In angiography, a user may more efficiently conduct surgery by using the X-ray apparatus according to the one or more of the above exemplary embodiments.

The X-ray apparatus according to the one or more of the above exemplary embodiments may track the target according to a user′ intention and generate the X-ray image or the fluoroscopy image without special manipulation by the user. Thus, the X-ray apparatus may provide a more efficient environment to the user.

The above-described embodiments of the present invention may be written as computer programs and may be implemented in general-use digital computers that execute the programs using a non-transitory computer-readable recording medium.

Examples of the non-transitory computer-readable recording medium include magnetic storage media (e.g., ROM, floppy disks, hard disks, etc.), optical recording media (e.g., CD-ROMs, or DVDs), etc), and transmission media such as Internet transmission media.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. An X-ray apparatus comprising:
a C-arm for adjusting a position of an X-ray source;
a table on which an object is positioned;
a data obtainer for obtaining position information of a target in the object; and
a controller for moving at least one of the C-arm and the table to allow tracking of the target based on the position information when capturing an X-ray image.

2. The X-ray apparatus of claim 1, wherein the controller moves at least one of the C-arm and the table to allow the target to be positioned within a predetermined distance from the center of the X-ray image based on the position information.

3. The X-ray apparatus of claim 1, wherein the controller moves at least one of the C-arm and the table to allow the target to be positioned in the center of the X-ray image based on the position information.

4. The X-ray apparatus of claim 1, further comprising: a user interface unit for receiving a first input selecting at least one of the C-arm and the table to be moved and setting an order of movements of the selected at least one of the C-arm and table,
wherein the controller moves at least one of the C-arm and the table to allow tracking of the target based on the first input and the position information when capturing the X-ray image.

5. The X-ray apparatus of claim 1, wherein,
the X-ray source emits an X-ray to the object,
further comprising: a detection unit for detecting the X-ray penetrating the object,
wherein the C-arm connects the X-ray source and the detection unit and adjusts positions of the X-ray source and the detection unit.

6. The X-ray apparatus of claim 1, wherein the target is a tip of a catheter.

7. The X-ray apparatus of claim 1, wherein the data obtainer obtains the position information of the target based on electrode signals detected from a plurality of electrodes attached to the object.

8. The X-ray apparatus of claim 7, wherein the controller sets first coordinates indicating a position of the target on a first coordinate system regarding the object based on the position information, transforms the first coordinates into second coordinates on a second coordinate system regarding the X-ray image, and moves at least one of the C-arm and the table to track the target based on the second coordinates.

9. The X-ray apparatus of claim 8, wherein the controller sets the first coordinate system as a 3-dimensional (3D) rectangular coordinate system based on electrode signals detected from a plurality of electrodes attached to positions corresponding to three axes that are perpendicular to each other, sets the second coordinate system as a 2D rectangular coordinate system that is a plane perpendicular to an irradiation direction of an X-ray, and sets a point on the plane closest to the first coordinates as the second coordinates.

10. The X-ray apparatus of claim 8, wherein the controller sets the first coordinate system as a 3D rectangular coordinate system based on electrode signals detected from a plurality of electrodes attached to positions corresponding to three axes that are perpendicular to each other, and sets the second coordinate system as a 3D rectangular coordinate system including an axis in the same direction as an irradiation direction of an X-ray.

11. The X-ray apparatus of claim 8, wherein the controller sets first coordinates indicating a position of the target on a first coordinate system regarding the object and transforms the first coordinates into second coordinates on a second coordinate system regarding the X-ray image based on an angle of the C-arm and the position information.

12. The X-ray apparatus of claim 7, wherein the data obtainer comprises a plurality of electrocardiogram (ECG) measurement electrodes attached to the object,
wherein the electrode signals are ECG signals.

13. The X-ray apparatus of claim 7, wherein the data obtainer measures impedance of the object included in an ROI based on the electrode signals and obtains the position information based on the impedance of the object.

14. The X-ray apparatus of claim 7, wherein the data obtainer measures impedance of the object included in an ROI based on the electrode signals, generates an impedance map of the object based on the impedance of the object, and obtains the position information based on the impedance map of the object.

15. The X-ray apparatus of claim 7, wherein the data obtainer obtains position information of the target in the object by image tracking the target appearing in the X-ray image.

16. (canceled)

17. A method of scanning an X-ray, the method comprising:
obtaining position information of a target in an object; and
moving at least one of a C-arm for adjusting a position of an X-ray source and a table on which the object is positioned to allow tracking of the target based on the position information when capturing an X-ray image.

18. The method of claim 17, wherein the moving of the at least one of the C-arm and table comprises: moving at least one of the C-arm and the table to allow the target to be positioned within a predetermined distance from the center of the X-ray image based on the position information.

19. The method of claim 17, wherein the moving of the at least one of the C-arm and table comprises: allowing the target to be positioned within a predetermined distance from the center of the X-ray image based on the position information, recognizing a boundary between the object and the target, and moving at least one of the C-arm and the table to track an ROI based on the boundary.

20. The method of claim 17, wherein the obtaining of the position information comprises: obtaining the position information of the target based on electrode signals detected from a plurality of electrodes attached to the object.

21. A non-transitory computer readable recording medium storing program for executing the method of claim 17.
